(19) United States
(12) Patent Application Publication
(10) Pub. No.: US 2012/0064012 A1
(43) Pub. Date: Mar. 15, 2012

(54) Uniform Fluorescent Microspheres with Hydrophobic Surfaces

(75) Inventor: Yu-Zhong Zhang, Eugene, OR (US)

(73) Assignee: LIFE TECHNOLOGIES CORPORATION, Carlsbad, CA (US)

(21) Appl. No.: 13/209,770

(22) Filed: Aug. 15, 2011

Related U.S. Application Data
(63) Continuation of application No. 11/772,063, filed on Jun. 29, 2007, now abandoned.
(60) Provisional application No. 60/806,382, filed on Jun. 30, 2006.

(51) Int. Cl. A61K 49/00

(52) U.S. Cl. 424/9.61; 424/9.6

(57) ABSTRACT

Fluorescent microspheres for the measurement of blood flow are provided. The microspheres are substantially uniform in diameter and have a hydrophobic surface, which allows them to circulate more freely throughout bloodstream, while reducing immunogenicity, particle aggregation and bioaccumulation. The hydrophobic surface on each microsphere is generally comprised of polymeric material having a limited surface charge.

### CROSS-REFERENCE TO RELATED APPLICATIONS

This application claims priority of U.S. Ser. No. 60/806,382, filed Jun. 30, 2006, which disclosure is herein incorporated by reference.

### FIELD OF THE INVENTION

This invention relates to fluorescent microspheres having a hydrophobic outer surface, as well as methods for their preparation. The invention also relates to methods for measuring blood flow by administering the fluorescent microspheres to a subject.

### BACKGROUND OF THE INVENTION

Detectable microspheres are useful tools as signaling tracers for blood flow measurements. After entering the animal's circulatory system, a signal from the microspheres can be detected through a proper imaging instrument and a range of characteristics of the blood flow in live animals can then be monitored and recorded for many different types of studies.

For some time, radioactively labeled microspheres have been used as tracers to estimate regional organ perfusion. In order to minimize radiation exposure and to reduce expensive storage and disposal of radioactive materials, the radiolabeled beads have generally been replaced with fluorescent microspheres.

Most of the existing applications of fluorescent microspheres in blood flow measurements are based on occlusion of capillaries (Van Oosterhout et al., J. Am. J Physiol. Heart Circ. Physiol, 275, H110-H115, 1998). This technology only measures the final distribution/accumulation of the microspheres by blood flow. The animal subject is typically sacrificed a few hours to few days after microsphere administration, and the tissues of interest (e.g. liver, lungs, etc.) are removed, sectioned, and analyzed for dye content associated with localized microspheres.

Additional applications include use of fluorescently stained microspheres in studies of blood flow within the microvasculature (intravital angiography) and for examination of the retinal blood supply system in live animals. One drawback of existing microspheres is their inability to freely flow through the blood stream without being obstructed by other particles and form aggregates therewith.

Accordingly, it is an object of the present invention to provide freely flowing, detectable microspheres for measurement of blood flow. Another object of the invention is to use the microspheres for diagnosis of diseases affecting the blood.

### SUMMARY OF THE INVENTION

Intravascular tracers of blood circulation can provide a description of the flow field over time and space. Described herein are fluorescent microspheres capable of providing detailed information regarding the intravascular flow field. The microspheres can maximize plasma half-life as well as minimize interactions with other blood components. The bioavailability of the particles in the blood circulation is improved by nanoscale size and low surface charge density. Intravital imaging of nanoparticles in the microcirculation demonstrated that the fluorescence intensity of the nanoparticles was a major determinant of both temporal and spatial resolution of the flow field. The microspheres described herein provide an accurate description of the localized intravascular flow field.

One aspect of the invention provides a method for measuring blood flow in a subject wherein the method comprises:

administering to the subject a plurality of microspheres, wherein the microspheres are impregnated with a dye having an excitation and emission spectrum compatible with in vivo or intravital imaging and further, wherein the microspheres have a hydrophobic outer surface;

illuminating the microspheres within the subject with an appropriate wavelength to form illuminated microspheres; and

observing the illuminated microspheres;

wherein, the blood flow is measured by detecting the movement of the microspheres in the subject.

In a more particular embodiment, the velocity of blood flow is measured by detecting movement of the microspheres.

In another embodiment, the plurality of microspheres are comprised of a first population having a substantially uniform first diameter. More particularly, the plurality of microspheres are comprised of a first population having a substantially uniform first diameter and a second population having a substantially uniform second diameter. In another more particular embodiment, the first population is impregnated with a different dye than the second population. In another embodiment, the first diameter is different than the second diameter, such as the first diameter is smaller than the second diameter. More particular still, the first diameter is 0.1 μm to 0.75 μm and the second diameter is 0.76 μm to 2 μm; or the first diameter is about 0.5 μm and the second diameter is about 1 μm.

In another more particular embodiment, said microspheres have a diameter of about 0.1 μm to 4 μm. More particular still, said microspheres have a diameter of about 0.2 μm to 2 μm. In another embodiment, said microspheres have a diameter of about 0.1 μm to 2 μm. In another embodiment, said microspheres have a diameter of about 0.01 μm to 10 μm. In another embodiment, said microspheres have a diameter of about 0.05 μm to 5 μm. In another embodiment, said microspheres have a diameter of about 0.25 μm to 2.5 μm. In another embodiment, said microspheres have a diameter of about 0.5 μm to 1 μm. In another embodiment, said microspheres have a diameter of less than 5 μm. In another embodiment, said microspheres have a diameter of less than 4 μm. In another embodiment, said microspheres have a diameter of less than 3 μm. In another embodiment, said microspheres have a diameter of less than 2 μm. In another embodiment, said microspheres have a diameter of less than 1 μm. In another embodiment, said microspheres have a diameter of about 0.01, 0.05, 0.075, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 μm.

In another more particular embodiment, said microspheres have a surface charge of less than 50 μEq/gram. More particularly, said microspheres have a surface charge of less than 10 μEq/gram. More particular still, said microspheres have a surface charge of less than 5 μEq/gram. In another embodiment, said microspheres have a surface charge of less than 100, 75, 60, 55, 45, 40, 35, 30, 25, 20, 15, 14, 13, 12, 11, 10, 9, 8, 7, 6, 5.5, 4.5, 4, 3.5, 3, 2.5, 2, 1.5, 1, 0.75, 0.5, 0.25, or 0.1 μEq/gram.

In another more particular embodiment, said microspheres have a diameter variation of 5% or less. More particularly, said microspheres have a diameter variation of 2% or less. More particular still, said microspheres have a diameter variation of 1% or less.

In another more particular embodiment, said hydrophobic outer surface is comprised of an organic polymer. More particularly, said hydrophobic outer surface is comprised of polymerized styrene moieties. More particular still, the styrene moieties are unsubstituted or substituted with chloromethyl groups.

In another more particular embodiment, said hydrophobic outer surface is a block co-polymer. In a more particular embodiment thereof, said block co-polymer is comprised of polyoxyethylene (PEO) and polyoxypropylene (PPO) moieties.

In another more particular embodiment, said microspheres are substantially free of aggregates. In another more particular embodiment, said microspheres are substantially free of aggregation with at least one of the following: leukocytes, erythrocytes, thrombocytes, serum proteins, electrolytes, carbohydrates, fats, or minerals.

In another more particular embodiment, said microspheres are administered to the subject parenterally. More particularly, said microspheres are administered intravenously to the subject. Alternatively, said microspheres are administered to the subject by inhalation.

In another more particular embodiment, the dye is selected from the group consisting of a pyrene, an anthracene, a naphthalene, an acridine, a stilbene, an indole, an oxazole, benzoxazole, a thiazole, a benzothiazole, a 4-amino-7-nitrobenz-2-oxa-1,3-diazole (NBD), a carbocyanine, a carbostyryl, a porphyrin, a salicylate, an anthranilate, an azulene, a perylene, a pyridine, a quinoline, a xanthene, an oxazine, a benzoxazine, a carbazine, a phenalenone, a coumarin, a benzofuran, a benzphenalenone, a semiconductor nanocrystal, and a fluorescent protein.

Another more particular aspect of the invention, further comprises the step of incubating said subject for a sufficient amount of time for the microspheres to circulate prior to illuminating the microspheres.

In another embodiment, the microspheres are imaged non-invasively.

In another more particular embodiment, the microspheres are used to monitor the pattern or turbulence of blood flow in the subject.

In another more particular embodiment, the subject is suffering from a disease associated with obstructed or abnormal blood flow.

In another more particular embodiment, the microspheres are bound to at least one other agent. More particularly, the agent is a protein. In another embodiment, the agent is an antibody, antibody fragment, polysaccharide, polynucleotide, receptor, or enzyme. In another more particular embodiment, the subject is suffering from a disease and the agent is capable of binding to a substrate specific for the disease. More particularly, the disease is selected from a viral infection, a bacterial infection, heart disease, cancer, ischemia, autoimmune disease, a CNS disorder, a metabolic disease, or a respiratory disease.

Another aspect of the invention provides a kit for measuring blood flow in a subject,

wherein the kit comprises:

a plurality of microspheres having, wherein the microspheres are impregnated with a dye having an excitation and emission spectrum compatible with in vivo or intravital imaging and further, wherein the microspheres have a hydrophobic outer surface;

packaging; and

written instructions on how to use the microspheres for the detection of blood flow.

Another more particular embodiment provides a syringe or inhalation device for administration of said microspheres to the subject. More particularly, the syringe is pre-packaged with said microspheres.

Another more particular embodiment provides a light source for excitation of the dye.

Another more particular embodiment provides a detector for measuring emissions released after excitation of the dye.

In another more particular embodiment, said microspheres have a diameter of about 0.1 μm to 4 μm. More particular still, said microspheres have a diameter of about 0.2 μm to 2 μm. In another embodiment, said microspheres have a diameter of about 0.1 μm to 2 μm. In another embodiment, said microspheres have a diameter of about 0.01 μm to 10 μm. In another embodiment, said microspheres have a diameter of about 0.05 μm to 5 μm. In another embodiment, said microspheres have a diameter of about 0.25 μm to 2.5 μm. In another embodiment, said microspheres have a diameter of about 0.5 μm to 1 μm. In another embodiment, said microspheres have a diameter of less than 5 μm. In another embodiment, said microspheres have a diameter of less than 4 μm. In another embodiment, said microspheres have a diameter of less than 3 μm. In another embodiment, said microspheres have a diameter of less than 2 μm. In another embodiment, said microspheres have a diameter of less than 1 μm. In another embodiment, said microspheres have a diameter of about 0.01, 0.05, 0.075, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 μm.

In another more particular embodiment, said microspheres have a surface charge of less than 50 μEq/gram. More particularly, said microspheres have a surface charge of less than 10 μEq/gram. More particular still, said microspheres have a surface charge of less than 5 μEq/gram. In another embodiment, said microspheres have a surface charge of less than 100, 75, 60, 55, 45, 40, 35, 30, 25, 20, 15, 14, 13, 12, 11, 10, 9, 8, 7, 6, 5.5, 4.5, 4, 3.5, 3, 2.5, 2, 1.5, 1, 0.75, 0.5, 0.25, or 0.1 μEq/gram.

In another more particular embodiment, said microspheres have a diameter variation of 5% or less. More particularly, said microspheres have a diameter variation of 2% or less. More particular still, said microspheres have a diameter variation of 1% or less.

In another more particular embodiment, said hydrophobic outer surface is comprised of an organic polymer. More particularly, said hydrophobic outer surface is comprised of polymerized styrene moieties. More particular still, the styrene moieties are unsubstituted or substituted with chloromethyl groups.

In another more particular embodiment, said hydrophobic outer surface is a block co-polymer. In a more particular embodiment thereof, said block co-polymer is comprised of polyoxyethylene (PEO) and polyoxypropylene (PPO) moieties.

In another more particular embodiment, said microspheres are substantially free of aggregates. In another more particular embodiment, said microspheres are substantially free of aggregation with at least one of the following: leukocytes, erythrocytes, thrombocytes, serum proteins, electrolytes, carbohydrates, fats, or minerals.

In another more particular embodiment, said microspheres are administered to the subject parenterally. More particularly, said microspheres are administered intravenously to the subject. Alternatively, said microspheres are administered to the subject by inhalation.

In another more particular embodiment, the dye is selected from the group consisting of a pyrene, an anthracene, a naphthalene, an acridine, a stilbene, an indole, an oxazole, benzoxazole, a thiazole, a benzothiazole, a 4-amino-7-nitrobenz-2-oxa-1,3-diazole (NBD), a carbocyanine, a carbostyryl, a porphyrin, a salicylate, an anthranilate, an azulene, a perylene, a pyridine, a quinoline, a xanthene, an oxazine, a benzoxazine, a carbazine, a phenalenone, a coumarin, a benzofuran, a benzphenalenone, a semiconductor nanocrystal, and a fluorescent protein.

In another more particular embodiment, the microspheres do not form aggregates in the subject.

In another more particular embodiment, the microspheres are used to monitor the pattern or turbulence of blood flow in the subject.

In another more particular embodiment, the subject is suffering from a disease associated with obstructed or abnormal blood flow. More particularly, the disease is selected from a viral infection, a bacterial infection, heart disease, cancer, ischemia, autoimmune disease, a CNS disorder, a metabolic disease, or a respiratory disease.

In one aspect of the invention, the microspheres are unsubstituted (i.e. not bound to an agent). This prevents aggregation and unwanted binding, permitting free flow through the blood stream.

In another more particular embodiment, the microspheres are bound to at least one other agent. More particularly, the agent is a protein. In another embodiment, the agent is an antibody, antibody fragment, polysaccharide, polynucleotide, receptor, or enzyme. In another more particular embodiment, the subject is suffering from a disease and the agent is capable of binding to a substrate specific for the disease. More particularly, the disease is selected from a viral infection, a bacterial infection, heart disease, cancer, ischemia, autoimmune disease, a CNS disorder, a metabolic disease, or a respiratory disease.

Another aspect of the invention provides a method for intravital imaging, comprising:

administering to the subject a plurality of microspheres having a substantially uniform diameter, wherein the microspheres are impregnated with a dye having an excitation and emission spectrum compatible with in vivo or intravital imaging and further, wherein the microspheres have a hydrophobic outer surface;

illuminating the microspheres within the subject with an appropriate wavelength; and

imaging emissions from subject.

In another more particular embodiment, the microspheres are imaged with an epifluorescence microscope.

In another more particular embodiment, said microspheres have a diameter of about 0.1 μm to 4 μm. More particular still, said microspheres have a diameter of about 0.2 μm to 2 μm. In another embodiment, said microspheres have a diameter of about 0.1 μm to 2 μm. In another embodiment, said microspheres have a diameter of about 0.01 μm to 10 μm. In another embodiment, said microspheres have a diameter of about 0.05 μm to 5 μm. In another embodiment, said microspheres have a diameter of about 0.25 μm to 2.5 μm. In another embodiment, said microspheres have a diameter of about 0.5 μm to 1 μm. In another embodiment, said microspheres have a diameter of less than 5 μm. In another embodiment, said microspheres have a diameter of less than 4 μm. In another embodiment, said microspheres have a diameter of less than 3 μm. In another embodiment, said microspheres have a diameter of less than 2 μm. In another embodiment, said microspheres have a diameter of less than 1 μm. In another embodiment, said microspheres have a diameter of about 0.01, 0.05, 0.075, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 μm.

In another more particular embodiment, said microspheres have a surface charge of less than 50 μEq/gram. More particularly, said microspheres have a surface charge of less than 10 μEq/gram. More particular still, said microspheres have a surface charge of less than 5 μEq/gram. In another embodiment, said microspheres have a surface charge of less than 100, 75, 60, 55, 45, 40, 35, 30, 25, 20, 15, 14, 13, 12, 11, 10, 9, 8, 7, 6, 5.5, 4.5, 4, 3.5, 3, 2.5, 2, 1.5, 1, 0.75, 0.5, 0.25, or 0.1 μEq/gram.

In another more particular embodiment, said microspheres have a diameter variation of 5% or less. More particularly, said microspheres have a diameter variation of 2% or less. More particular still, said microspheres have a diameter variation of 1% or less.

In another more particular embodiment, said hydrophobic outer surface is comprised of an organic polymer. More particularly, said hydrophobic outer surface is comprised of polymerized styrene moieties. More particular still, the styrene moieties are unsubstituted or substituted with chloromethyl groups.

In another more particular embodiment, said hydrophobic outer surface is a block co-polymer. In a more particular embodiment thereof, said block co-polymer is comprised of polyoxyethylene (PEO) and polyoxypropylene (PPO) moieties.

In another more particular embodiment, said microspheres are substantially free of aggregates. In another more particular embodiment, said microspheres are substantially free of aggregation with at least one of the following: leukocytes, erythrocytes, thrombocytes, serum proteins, electrolytes, carbohydrates, fats, or minerals.

In another more particular embodiment, said microspheres are administered to the subject parenterally. More particularly, said microspheres are administered intravenously to the subject. Alternatively, said microspheres are administered to the subject by inhalation.

In another more particular embodiment, the dye is selected from the group consisting of a pyrene, an anthracene, a naphthalene, an acridine, a stilbene, an indole, an oxazole, benzoxazole, a thiazole, a benzothiazole, a 4-amino-7-nitrobenz-2-oxa-1,3-diazole (NBD), a carbocyanine, a carbostyryl, a porphyrin, a salicylate, an anthranilate, an azulene, a perylene, a pyridine, a quinoline, a xanthene, an oxazine, a benzoxazine, a carbazine, a phenalenone, a coumarin, a benzofuran, a benzphenalenone, a semiconductor nanocrystal, and a fluorescent protein.

Another more particular aspect of the invention, further comprises the step of incubating said subject for a sufficient amount of time for the microspheres to circulate prior to illuminating the microspheres.

In another more particular embodiment, the microspheres are used to monitor the pattern or turbulence of blood flow in the subject.

In another more particular embodiment, the subject is suffering from a disease associated with obstructed or abnormal blood flow.

In another more particular embodiment, the microspheres are bound to at least one other agent. More particularly, the agent is a protein. In another embodiment, the agent is an antibody, antibody fragment, polysaccharide, polynucleotide, receptor, or enzyme. In another more particular embodiment, the subject is suffering from a disease and the agent is capable of binding to a substrate specific for the disease. More particularly, the disease is selected from a viral infection, a bacterial infection, heart disease, cancer, ischemia, autoimmune disease, a CNS disorder, a metabolic disease, or a respiratory disease.

Another aspect of the invention provides a method for identifying a subject suffering from a disease affecting the blood, comprising:

administering to the subject a plurality of microspheres, wherein the microspheres are impregnated with a dye having an excitation and emission spectrum compatible with in vivo or intravital imaging and further, wherein the microspheres have a hydrophobic outer surface;

illuminating the microspheres within the subject with an appropriate wavelength to form illuminated microspheres; and

observing the illuminated microspheres;

wherein, the disease affects the movement of said microspheres within the subject.

In another more particular embodiment, said microspheres have a diameter of about 0.1 μm to 4 μm. More particular still, said microspheres have a diameter of about 0.2 μm to 2 μm. In another embodiment, said microspheres have a diameter of about 0.1 μm to 2 μm. In another embodiment, said microspheres have a diameter of about 0.01 μm to 10 μm. In another embodiment, said microspheres have a diameter of about 0.05 μm to 5 μm. In another embodiment, said microspheres have a diameter of about 0.25 μm to 2.5 μm. In another embodiment, said microspheres have a diameter of about 0.5 μm to 1 μm. In another embodiment, said microspheres have a diameter of less than 5 μm. In another embodiment, said microspheres have a diameter of less than 4 μm. In another embodiment, said microspheres have a diameter of less than 3 μm. In another embodiment, said microspheres have a diameter of less than 2 μm. In another embodiment, said microspheres have a diameter of less than 1 μm. In another embodiment, said microspheres have a diameter of about 0.01, 0.05, 0.075, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 μm.

In another more particular embodiment, said microspheres have a surface charge of less than 50 μEq/gram. More particularly, said microspheres have a surface charge of less than 10 μEq/gram. More particular still, said microspheres have a surface charge of less than 5 μEq/gram. In another embodiment, said microspheres have a surface charge of less than 100, 75, 60, 55, 45, 40, 35, 30, 25, 20, 15, 14, 13, 12, 11, 10, 9, 8, 7, 6, 5.5, 4.5, 4, 3.5, 3, 2.5, 2, 1.5, 1, 0.75, 0.5, 0.25, or 0.1 μEq/gram.

In another more particular embodiment, said microspheres have a diameter variation of 5% or less. More particularly, said microspheres have a diameter variation of 2% or less. More particular still, said microspheres have a diameter variation of 1% or less.

In another more particular embodiment, said hydrophobic outer surface is comprised of an organic polymer. More particularly, said hydrophobic outer surface is comprised of polymerized styrene moieties. More particular still, the styrene moieties are unsubstituted or substituted with chloromethyl groups.

In another more particular embodiment, said hydrophobic outer surface is a block co-polymer. In a more particular embodiment thereof, said block co-polymer is comprised of polyoxyethylene (PEO) and polyoxypropylene (PPO) moieties.

In another more particular embodiment, said microspheres are substantially free of aggregates. In another more particular embodiment, said microspheres are substantially free of aggregation with at least one of the following: leukocytes, erythrocytes, thrombocytes, serum proteins, electrolytes, carbohydrates, fats, or minerals.

In another more particular embodiment, said microspheres are administered to the subject parenterally. More particularly, said microspheres are administered intravenously to the subject. Alternatively, said microspheres are administered to the subject by inhalation.

In another more particular embodiment, the dye is selected from the group consisting of a pyrene, an anthracene, a naphthalene, an acridine, a stilbene, an indole, an oxazole, benzoxazole, a thiazole, a benzothiazole, a 4-amino-7-nitrobenz-2-oxa-1,3-diazole (NBD), a carbocyanine, a carbostyryl, a porphyrin, a salicylate, an anthranilate, an azulene, a perylene, a pyridine, a quinoline, a xanthene, an oxazine, a benzoxazine, a carbazine, a phenalenone, a coumarin, a benzofuran, a benzphenalenone, a semiconductor nanocrystal, and a fluorescent protein.

Another more particular aspect of the invention, further comprises the step of incubating said subject for a sufficient amount of time for the microspheres to circulate prior to illuminating the microspheres.

In another more particular embodiment, the microspheres are used to monitor the pattern or turbulence of blood flow in the subject.

In another more particular embodiment, the subject is suffering from a disease associated with obstructed or abnormal blood flow.

In another more particular embodiment, the microspheres are bound to at least one other agent. More particularly, the agent is a protein. In another embodiment, the agent is an antibody, antibody fragment, polysaccharide, polynucleotide, receptor, or enzyme. In another more particular embodiment, the subject is suffering from a disease and the agent is capable of binding to a substrate specific for the disease. More particularly, the disease is selected from a viral infection, a bacterial infection, heart disease, cancer, ischemia, autoimmune disease, a CNS disorder, a metabolic disease, or a respiratory disease.

Another aspect of the invention provides a microsphere or plurality of microspheres as described herein and blood. Another aspect of the invention provides a microsphere or plurality of microspheres as described herein and leukocytes and/or erythrocytes.

Further embodiments of the invention include those described in the detailed description.

### BRIEF DESCRIPTION OF THE FIGURES

FIG. 1: TEM image of 500 nm green fluorescent, hydrophobic polystyrene nanospheres indicates the particles are not aggregated and are highly uniform in size with a standard variation of diameter less than 3%.

FIG. 2: Measured flow velocities of the particles (500 nm, 2 um, and 4 um) and PKH26 labeled red blood cells in the flow chamber. Particle tracking was performed on 1000 frame image stacks obtained at 60-70 fps. A minimum of 100 particles/red cells were tracked at each flow rate. Error bars reflect one standard deviation.

FIG. 3: Time-position tracking of 500 nm particles at 4 flow velocities: A) 500 um/sec, B) 1000 um/sec, C) 2000 um/sec and D) 4000 um/sec. The flow paths of 8 randomly selected 500 nm particles are shown at each flow velocity. The position of the nanoparticles at 10 msec intervals are noted with an ‘x’; the arrows show the direction of flow.

FIG. 4: Intravital micrograph and time-position tracking of 500 nm particles passing through the ear microcirculation. A) A selected image obtained 5 minutes after the intravenous tail vein injection of 500 nm particles. A single nanoparticle is shown (arrow; bar=120 um). B) Representative flow paths of two consecutive 500 nm particles. Nanoparticle positions and ◯) were recorded at 33 msec/sec intervals.

FIG. 5: Intravital microscopy of BODIPY-green nanoparticles (500 nm) in the mouse colon shown at two magnifications (A-C bar ¼ 50 lm; D-F bar ¼ 200 lm). A,D: Single frames demonstrating nanoparticles (rings) in the blood circulation. Tissue autofluorescence is noted (arrow). B,E: The flow paths of two randomly chosen particles is shown in red and yellow. Nanoparticle position is plotted at 16 ms intervals; note the variability in instantaneous velocity. C,F: Timespace image of the same area of the circulation obtained by digitally combining 250 consecutive images. The resulting image reveals vessels previously unrecognized (arrows) based on tissue autofluorescence. Digital recombination of the 250 frame video sequence provides an integrated measure of nanoparticle flux. The intravascular injection of highly fluorescent biocompatible nanoparticles provides an opportunity to define the flow field in the blood circulation (FIGS. 5A and 5D). Individual particles can be tracked F6 providing time-space plots of the flow path (FIGS. 5B and 5E). In addition, many flow paths obtained over time can be digitally combined to provide an integrated assessment of flow in the system (FIGS. 5C and 5F). These data suggest that nanoparticles-prepared with improved biocompatibility and fluorescence characteristics provide useful insights into biological flow fields.

FIG. 6 is a schematic of a videomicroscopy system used herein. In addition to the inverted microscope, the system includes a fiberoptic light guide, an EMCCD camera as well as computer controlled dual excitation and emission filter wheels. In this system, the streaming images are acquired to 4 gb random access memory (RAM) and written to redundant array of independent disks (RAID) controlled hard drives (15,000 rpm). The image stacks were acquired, distance calibrated and timestamped by the MetaMorph software program based on the Xeon processor system bus clock.

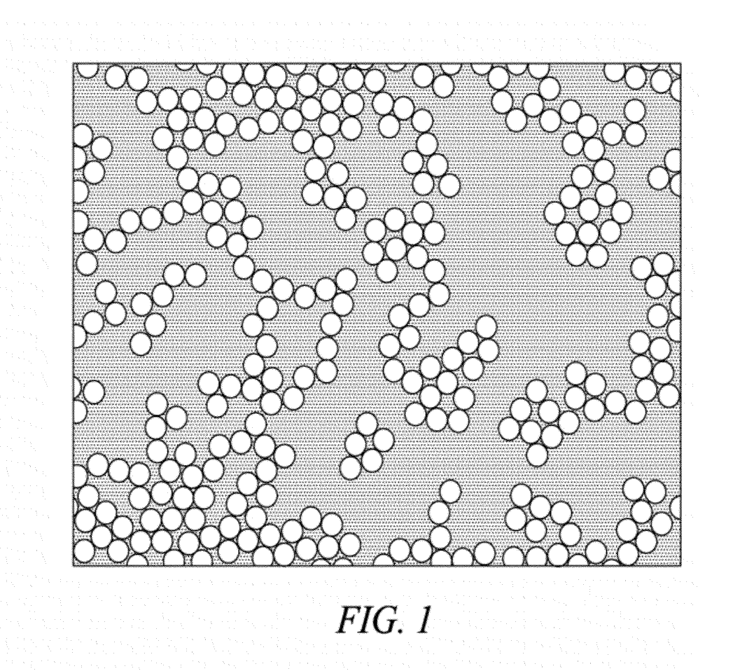

### DETAILED DESCRIPTION OF THE INVENTION

### Introduction

The present invention provides novel formulations for in vivo and intravital imaging. These formulations include polymeric microspheres (also referred to herein as microparticles) that have been stained with a fluorescent dye(s) having an excitation and emission wavelength compatible with in vivo or intravital imaging, typically about 300 nm to about 800 nm, and that have a hydrophobic outer surface, such as an unsubstituted polystyrene matrix. Alternatively, the microspheres can be coated with a block copolymer (also herein referred to as a surfactant). The coated microspheres travel relatively freely within the circulating blood.

The present hydrophobic microspheres and their use for in vivo or intravital imaging have many advantages compared to known contrast agents, and in a preferred embodiment these advantages, include, but are not limited to:
Polystyrene microspheres in the size range (100 to 2000 nm diameter) have no known intrinsic toxicity and are likely to be non-immunogenic.
Each particle contains many dye molecules and emits light intensely
The impregnated dye is protected within the polymer sphere from chemical and photodegradation.
A wide range of sizes, surface treatments and emission wavelengths can be easily prepared.
Emulsion polymerization results in highly uniform spheres and a high degree of structural and functional homogeneity.
The in vivo or intravital images produced demonstrate a high degree of unencumbered circulation within the blood, which provides an accurate depiction of the velocity of blood flow (higher resolution than other commercially available contrast agents based on dye-labeled macromolecules).
The inert nature of the dye impregnated microspheres leads to long in vivo residence times and thus increased imaging times for blood flow velocity determinations.
Reduced aggregation with various biological particles, such as leukocytes, erythrocytes, thrombocytes, serum proteins, electrolytes, carbohydrates, fats, or minerals.
Vascularized microspheres in a wide range of colors and sizes can be prepared using relatively well-known materials and processing methods.
Very bright images with much higher intrinsic contrast are possible with the present formulation than have been previously observed with available optical contrast agents.
Unique applications such as monitoring or grading disease states can be envisioned using these uniquely uniform particles.
These materials could be acceptable for both animal and human imaging due to their low toxicity.

Thus, the present fluorescent microspheres function as improved highly effective blood flow velocity indicators, which can prove useful in a variety of different applications, including diagnosis of diseases affecting the vasculature.

Blood is an aqueous multifunctional fluid with large number of cells and high concentration of serum proteins and other components. All these proteins and cells have hydrophilic surfaces. When microspheres with high surface charge content (either positive or negative) are introduced in to blood stream, their hydrophilic surface will allow the particles to quickly interact with blood components.

The current invention provides microspheres that greatly reduce non-specific interaction of the microspheres with blood components and the surface of blood vessels. The microspheres have much less chance to stick to blood cells or platelets, or to be entrapped in epithelial cell junctions of blood vessels. They travel relatively freely within the circulating blood. As a result, the circulation time of the fluorescent microspheres is considerably increased. In addition, the floating patterns of microspheres more closely reflect the true status/conditions of blood flow.

In one embodiment, the present invention involves polystyrene microspheres containing an internal fluorescent dye or dye combination; or a mixture of fluorescent polystyrene microspheres containing different dyes. The particles are highly uniformed in size, with size coefficient of variation usually less than 5%, or less than 4%, or less than 3%, or less than 2%, or even less than 1%. More particularly, the surfaces of the fluorescent microspheres are specially treated to have low surface charge content, which can be accomplished through either chemical modifications or physical coatings of other biological and/or synthetic molecules such as non-ionic surfactants. An example is Pluronic® F-127, a block copolymer surfactant that is nonionic and nontoxic. Accordingly, the fluorescent microspheres of the present invention have a hydrophobic surface and high degree of functional homogeneity.

The methods described herein provide an improved means to achieve sensitive detection of flow velocity and flow pattern of blood circulation in live animals. The microspheres of the present invention circulate inside of blood vessels of an animal, after administration, such as by intravenous injection or inhalation, for a much longer time period without noticeable reduction in bead numbers or the accumulation of beads on the surface of blood vessels. They also have no intrinsic toxicity and have reduced or no immunogenicity.

One particular embodiment invention provides a batch of 1 μm sulfate beads, as example, that has only 3.4 μEq/gram surface charge content, which is almost 167 times less than comparable CML (carboxylate modified) beads, which have a 503 μEq/gram of total surface negative charge content. The microspheres of the present invention have up to 90% or more hydrophobic surface area (polymerized styrenes), that acts likes a “non-reactive” protection layer for the microspheres. When the hydrophobic beads get in to blood circulation, the hydrophobic surface “layer” prevents or minimizes the interaction of fluorescent microsphere with: erythrocytes (red blood cells), leukocytes (white blood cells), thrombocytes (platelets), serum proteins, electrolytes, carbohydrates, fats and minerals.

Accordingly, one embodiment of the invention provides hydrophobic microspheres with a low surface charge. Particularly, the microspheres have a surface charge of less than 200 μEq/gram, or less than 100 μEq/gram, or less than 50 μEq/gram, or less than 25 μEq/gram, or less than 20 μEq/gram, less than 15 μEq/gram, or less than 10 μEq/gram, or less than 9 μEq/gram, or less than 8 μEq/gram, or less than 7 μEq/gram, or less than 6 μEq/gram, or less than 5 μEq/gram, or less than 4 μEq/gram, or less than 3 μEq/gram, or less than 2 μEq/gram, or less than 1 μEq/gram. In another embodiment, the microspheres have a surface charge of between 0.1 to 200 μEq/gram, or 0.5 to 100 μEq/gram, or 0.75 to 50 μEq/gram, or 1 to 10 μEq/gram, or 1 to 5 μEq/gram, or 2 to 4 μEq/gram.

The hydrophilic non-reactive layer allows the beads having smoother surface, traveling more freely, having less chance of stick together or packed on the luminal surface of blood vessels. Therefore the microspheres with low surface charge content have longer circulation time.

Additionally, size-selection of fluorescent microspheres is an important consideration in particle-tracking based blood flow velocity and pattern measurements. The signaling particles should be large enough to produce enough optical signals visible above background noise. On the other hand, the particles should be small enough compared to the dimensions of the blood vessels and should be much smaller in size than the distance over which significant spatial velocity gradients are to be measured. Large particle images may limit the spatial resolution of the velocity measurements.

Accordingly, in another embodiment of the invention, the microspheres have a diameter between 10 nm to 10 μm, or 100 nm to 5 μm, or 250 nm to 2.5 μm, or 500 nm to 1 μm.

Finally, the particles for each test should be highly uniform in size distribution, amongst individual populations, so their dynamic behaviors in blood flow remain the same during measurements. To meet these criterions, microspheres having a diameter with less than 10% size variation, or less than 9% size variation, or less than 8% size variation, or less than 7% size variation, or less than 6% size variation, or less than 5% size variation, or less than 4% size variation, or less than 3% size variation, or less than 2% size variation, or less than 1% size variation, or less than 0.5% size variation, are provided.

Careful selection of fluorescent microspheres at proper size range, and highly uniformed in size and intensity, helps to gain more accurate measurement of blood flow/turbulence. For example, the blood flow at the normal carotid bifurcation is highly turbulent and prone to aggregation by various molecules. In particular, microspheres with high surfaces charges are accumulated at the junction of the carotid bifurcation, thereby inaccurately reflecting the turbulence and blood flow at that point. It can however be precisely recorded using the fluorescent microspheres described in this invention, which will provide more valuable, accurate information for physiological study and pharmacological studies.

The emission signals of the microspheres within the blood circulation in a living animal are observed using intravital imaging videomicroscope capable of exciting fluorescent dyes in the microspheres and detecting the signals/images; the signals/images are digitally recorded at a high frame per second rate.

Subsequent analysis of series of video images of microspheres provides reliable information on the characteristics of blood flow such as flow direction, velocity, distribution of velocity, acceleration, vortices and pulsation.

Microspheres of the present invention also help to acquire high resolution microscopic video images of blood flow in heart of a growing embryonic Zebrafish. Since the volume and velocity of blood flow is crucial for the proper development of infant heart, developing accurate measurement of blood flow velocity is very important part of development studies. A better understanding of the mechanisms of blood flow during different developmental stages might lead to advanced early-treatments to prevent certain types of heart diseases in human.

Therefore, the microspheres of the present invention have great utility in intravital imaging application for blood flow research. Notably, the cellular composition of the microcirculation creates blood flow that can be unsteady and non-uniform. To obtain information about non-uniform cellular trajectories, in vivo and intravital imaging techniques provide both detailed tracking of individual particles as well as an approach to simultaneous multi-color particle tracking Particularly relevant to biologic systems, lagrangian methods provide information about the fate of individual particles and flow in the system.

Furthermore, the microspheres of the present invention may be administered to the subject orally, parenterally, sublingually, by aerosolization or inhalation, rectally, intracisternally, intravaginally, intraperitoneally, bucally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrasternal injection, or infusion techniques.

Methods of formulation are well known in the art and are disclosed, for example, in Remington: The Science and Practice of Pharmacy, Mack Publishing Company, Easton, Pa., 19th Edition (1995). Compositions for use in the present invention can be in the form of sterile, non-pyrogenic liquid solutions or suspensions, coated capsules, suppositories, lyophilized powders, transdermal patches or other forms known in the art.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-propanediol or 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the microspheres' half-life, it may be desirable to slow the absorption from subcutaneous or intramuscular injection. This may be accomplished by the use of a liquid suspension of crystalline or amorphous material with poor water solubility. The rate of absorption of the drug then depends upon its rate of dissolution which, in turn, may depend upon particle size and form. Alternatively, delayed absorption of a parenterally administered drug form may be accomplished by dissolving or suspending the microspheres in an oil vehicle.

Solid dosage forms for oral administration include capsules, tablets, pills, powders, and granules. Liquid dosage forms for oral administration include pharmaceutically acceptable emulsions, microemulsions, solutions, suspensions, syrups and elixirs. In addition to the microspheres, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Compositions of the invention may also be formulated for delivery as a liquid aerosol or inhalable dry powder. Liquid aerosol formulations may be nebulized predominantly into particle sizes that can be delivered to the terminal and respiratory bronchioles. Aerosolized formulations of the invention may be delivered using an aerosol forming device, such as a jet, vibrating porous plate or ultrasonic nebulizer. Further, the formulation preferably has balanced osmolarity ionic strength and chloride concentration, and the smallest aerosolizable volume able to deliver effective dose of the microspheres of the invention to the site of interest. Additionally, the aerosolized formulation preferably does not impair negatively the functionality of the airways and does not cause undesirable side effects.

Aerosolization devices suitable for administration of aerosol formulations of the invention include, for example, jet, vibrating porous plate, ultrasonic nebulizers and energized dry powder inhalers. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AERONEB and AERODOSE vibrating porous plate nebulizers (AeroGen, Inc., Sunnyvale, Calif.), SIDESTREAM nebulizers (Medic-Aid Ltd., West Sussex, England), PARI LC and PARI LC STAR jet nebulizers (Pari Respiratory Equipment, Inc., Richmond, Va.), and AEROSONIC (DeVilbiss Medizinische Produkte (Deutschland) GmbH, Heiden, Germany) and ULTRAAIRE (Omron Healthcare, Inc., Vernon Hills, Ill.) ultrasonic nebulizers.

### DEFINITIONS

It must be noted that, as used in this specification and the appended claims, the singular form “a”, “an” and “the” include plural referents unless the context clearly dictates otherwise. Thus, for example, reference to “a present compound” includes a plurality of compounds and reference to “a fluorophore” includes a plurality of ions and the like.

Unless defined otherwise, all technical and scientific terms used herein have the same meaning as commonly understood by one of ordinary skill in the art to which this invention is related. The following terms are defined for purposes of the invention as described herein.

Reference to “microspheres” and “microparticles” indicates both micrometer-sized and nanometer-sized spheres. The microspheres of the present invention are spherical in shape and generally have uniform diameters of between 100 nm and 4 μm. The microspheres have hydrophobic surfaces which reduces interactions with nearby particles.

Reference to “hydrophobic outer surface” as used herein indicates a low surface charge. The term “outer surface” in no way limits the substance of the inner core material which may be comprised of the same or different material as the outer surface.

The “diameter” of a microsphere is the maximum distance between two antipodal points on the surface of the sphere. “Substantially uniform in diameter” refers to a diameter variation between particles or microspheres of less than about 5%.

A “subject” includes any animal, such as a human, monkey, rat, mouse, dog, cat, or fish, including a zebrafish.

The term “aqueous solution” as used herein refers to a solution that is predominantly water and retains the solution characteristics of water. Where the aqueous solution contains solvents in addition to water, water is typically the predominant solvent.

The term “buffer” as used herein refers to a system that acts to minimize the change in acidity or basicity of the solution against addition or depletion of chemical substances.

The term “dye” as used herein refers to a compound or particles that emit light to produce an observable detectable signal. “Dye” includes fluorescent and nonfluorescent agents that include without limitations pigments, fluorophores, chemiluminescent compounds, luminescent compounds and chromophores. The term “fluorophore” as used herein refers to a compound that is inherently fluorescent or demonstrates a change in fluorescence upon binding to a biological compound or metal ion, i.e., fluorogenic. Numerous dyes are known to those skilled in the art and are described herein, and include, but are not limited to, semiconductor nanocrystals, coumarin, acridine, furan, indole, quinoline, cyanine, benzofuran, quinazolinone, benzazole, borapolyazaindacene and xanthenes, with the latter including fluoroscein, rhodamine, rhodol, rosamine and derivatives thereof as well as other fluorescent dyes described in INVITROGEN, THE HANDBOOK, A GUIDE TO FLUORESCENT PROBES AND LABELING by Richard P. Haugland (10^{th} edition, 2005). Accordingly, reference to a particular group or scaffold, such as “indole” refers to a composition comprising that moiety and optionally additional substituents.

The term “kit” as used herein refers to a packaged set of related components, typically one or more compounds or compositions.

The term “sample” as used herein refers to any material that may contain an analyte of interest and is intended to include the term in its broadest sense. Suitable samples include, but are not limited to, recombinant proteins over expressed in cells that are in the form of inclusion bodies or secreted from cells, normal and diseased cells, cell homogenates (cell lysates); cell fractions; tissue homogenates (tissue lysates); immunoprecipitates, biological fluids, such as blood, urine and cerebrospinal fluid; tears; feces; saliva; and lavage fluids, such as lung or peritoneal lavages. Typically, the sample is a cell extract or a biological fluid that comprises endogenous host cell proteins or expressed recombinant proteins.

The term “detectable response” as used herein refers to a change in or an occurrence of, a signal that is directly or indirectly detectable either by observation or by instrumentation and the presence or magnitude of which is a function of the presence of a target in the test sample. Typically, the detectable response is an optical response resulting in a change in the wavelength distribution patterns or intensity of absorbance or fluorescence or a change in light scatter, fluorescence quantum yield, fluorescence lifetime, fluorescence polarization, a shift in excitation or emission wavelength or a combination of the above parameters. The detectable change in a given spectral property is generally an increase or a decrease. However, spectral changes that result in an enhancement of fluorescence intensity and/or a shift in the wavelength of fluorescence emission or excitation are also useful.

The term “illuminating” as used herein refers to the application of any light source, including near-infrared (NIR) and visible light, capable of exciting dyes impregnated within the microspheres of the invention.

The term “in vivo imaging” as used herein refers to methods or processes in which the structural, functional, or physiological state of a living being is examinable.

The term “intravital imaging” as used herein refers to methods or processes for capturing images in live animals. In a preferred embodiment, the intravital imaging provides a measure of velocity of fluorescent microspheres circulating within the vasculature of a subject.

The term “non invasive in vivo imaging” as used herein refers to methods or processes in which the structural, functional, or physiological state of a being is examinable by remote physical probing without the need for breaching the physical integrity of the outer (skin) or inner (accessible orifices) surfaces of the body.

Reference to “blood flow” indicates the movement of blood throughout the body or in specific locations. Blood flow includes speed, velocity, turbulence or any other attribute of blood movement.

The term “vasculature” as used herein refers to the network of blood vessels in a subject.

### Suitable Microspheres

Preferably the microspheres of the present invention are hydrophobic and substantially uniform; that is for a given batch of microspheres, the individual microspheres within the batch will be essentially identical and contain a low surface charge. The uniformity is typically measured using the standard deviation in diameters, or by the coefficient of variation (CV). The coefficient of variation for the microspheres of the invention is typically about 1-3%, depending upon the size of the particular microspheres.

In a particular aspect, the present microsphere-based contrast agent contains highly size-uniform emulsion-polymerized polystyrene microspheres that comprise a fluorescent dye incorporated within the microsphere. A wide variety of different microspheres may be utilized in the present invention. Preferably, the microspheres (which are small spheres stained with a fluorescent dye having an excitation and emission spectra between about 300 nm to about 800 nm), are composed of biocompatible synthetic polymers or copolymers prepared from monomers such as, but not limited to, acrylic acid, methacrylic acid, ethyleneimine, crotonic acid, acrylamide, ethyl acrylate, methyl methacrylate, 2-hydroxyethyl methacrylate (HEMA), lactic acid, glycolic acid, ε-caprolactone, acrolein, cyanoacrylate, bisphenol A, epichlorhydrin, hydroxyalkylacrylates, siloxane, dimethylsiloxane, ethylene oxide, ethylene glycol, hydroxyalkyl-methacrylates, N-substituted acrylamides, N-substituted methacrylamides, N-vinyl-2-pyrrolidone, 2,4-pentadiene-1-ol, vinyl acetate, acrylonitrile, styrene, p-amino-styrene, p-amino-benzyl-styrene, sodium styrene sulfonate, sodium 2-sulfoxyethylmethacrylate, vinyl pyridine, aminoethyl methacrylates, 2-methacryloyloxy-trimethylammonium chloride, and polyvinylidene, as well polyfunctional crosslinking monomers such as N,N′-methylenebisacrylamide, ethylene glycol dimethacrylates, 2,2′-(p-phenylenedioxy)-diethyl dimethacrylate, divinylbenzene, triallylamine and methylenebis-(4-phenyl-isocyanate), including combinations thereof. In one aspect the polymers include polyacrylic acid, polyethyleneimine, polymethacrylic acid, polymethylmethacrylate, polysiloxane, polystyrene, polydimethylsiloxane, polylactic acid, poly(ε-caprolactone), epoxy resin, poly(ethylene oxide), poly(ethylene glycol), and polyamide (nylon). In another aspect the copolymers include the following: polyvinylidene-polyacrylonitrile, polyvinylidene-polyacrylonitrile-polymethylmethacrylate, and polystyrene-polyacrylonitrile. A most preferred polymer is polystyrene.

The term biocompatible, as used herein in conjunction with the terms monomer or polymer, is employed in its conventional sense, that is, to denote polymers that do not substantially interact with the tissues, fluids and other components of the body in an adverse fashion in the particular application of interest, such as the aforementioned monomers and polymers.

Examples of select microspheres known in the art include, but are not limited to, poly(D,L-lactide-co-glycolide) (PLGA) microspheres; poly(epsilon-caprolactone) (PCL) microspheres; poly(D,L-lactide)/poly(D,L-lactide-co-glycolide) composite microparticles; alginate-poly-L-lysine alginate (APA) microcapsules; alginate microspheres; poly(D,L-lactic-co-glycolic acid) microspheres; chitosan microspheres; poly[p-(carboxyethylformamido)-benzoic anhydride] (PCEFB) microspheres; Hyaluronan-based microspheres; biodegradable microspheres; microspheres of PMMA-PCL-cholesterol; poly(propylene fumarate)/poly(lactic-co-glycolic acid) blend microspheres; poly(lactide-co-glycolide acid-glucose) microspheres; polylactide co-glycolide (PLG) microspheres; poly(methacrylic acid) microspheres; poly(methylidene malonate 2.1.2)-based microspheres; ammonio methacrylate copolymer microspheres; poly(ethylene oxide)-modified poly(beta-amino ester) microspheres; methoxy poly(ethylene glycol)/poly(epsilon-caprolactone) microspheres; polyferrocenylsilane microspheres; poly(fumaric-co-sebacic acid) (P(FASA)) microspheres; poly(lactide-co-glycolide) and poly(ethylene oxide)-poly(propylene oxide)-poly(ethylene oxide) triblock copolymer microspheres; polyisobutylcyanoacrylate microspheres; polystyrene core-glycopolymer corona nanospheres; poly(ethyl-2-cyanoacrylate) microspheres; POE-PEG-POE triblock copolymeric microspheres; poly(DL-lactide) microparticles; albumin microspheres; poly(EGDMA/HEMA) based microbeads; glutaraldehyde crosslinked sodium alginate microbeads; pectin microspheres; methoxy poly(ethylene glycol) and glycolide copolymer microspheres; crosslinked polyethyleneimine microspheres; poly(glycidyl methacrylate-co-ethylene dimethacrylate); cellulose acetate trimellitate ethylcellulose blend microspheres; poly(ester) microspheres; polyacrylamide microcarriers; polyacrolein microspheres; 2-hydroxyethyl methacrylate microspheres The microspheres may be of varying size. Suitable size microspheres include those ranging from between about 10 and about 5000 nm in outside diameter, preferably between about 50 and about 500 nm in outside diameter. Most preferably, the microspheres are about 75 nm to about 200 nm in outside diameter.

The microspheres of the invention may be prepared by various processes, as will be readily apparent to those skilled in the art, such as by interfacial polymerization, phase separation and coacervation, multiorifice centrifugal preparation, and solvent evaporation, or a combination thereof. Suitable procedures which may be employed or modified in accordance with the present disclosure to prepare microspheres within the scope of the invention include those procedures disclosed in U.S. Pat. Nos. 4,179,546; 3,945,956; 4,108,806; 3,293,114; 3,401,475; 3,479,811; 3,488,714; 3,615,972; 4,549,892; 4,540,629; 4,421,562; 4,420,442; 4,898,734; 4,822,534; 3,732,172; 3,594,326; 3,015,128; Deasy, Microencapsulation and Related Drug Processes, Vol. 20, Chs. 9 and 10, pp. 195-240 (Marcel Dekker, Inc., N.Y., 1984), Chang et al., Canadian J. of Physiology and Pharmacology, Vol 44, pp. 115-129 (1966), and Chang, Science, Vol. 146, pp. 524-525 (1964).

Using controlled emulsion polymerization followed by fluorescent dye impregnation, microspheres with a wide range of sizes, surfaces and optical properties can be produced. Although a relatively mature technology in terms of manufacturing and handling, the use of latex or polystyrene microspheres for formulating in vivo or intravital contrast imaging agents, and in particular using microspheres impregnated with NIR fluorescent dyes for this purpose, to the best of our knowledge has never been used before.

Unstained microspheres in a variety of sizes and polymer compositions that are suitable for preparation of fluorescent microspheres of the invention are available from a variety of sources, including: Interfacial Dynamics Corporation (Portland, Oreg.), Bangs Laboratories (Carmel, Ind.), Dynal (Great Neck, N.Y.), Polysciences (Warrington, Pa.), Seradyne (Indianapolis, Ind.), Magsphere (Pasadena, Calif.), Duke Scientific Corporation (Palo Alto, Calif.), Spherotech Inc. (Libertyville, Ill.) and Rhone-Poulenc (Paris, France). Chemical monomers for preparation of microspheres are available from numerous sources.

### Coated Microspheres

To coat polystyrene microsphere surfaces in the working formulation of the instant invention, poloxamers block copolymers of ethylene oxide and propylene oxide can be used, generally having a molecular weight within the range of 1000 to 16,000, and of the structure:

HO(C₂H₄O)_{b}(C₃H₆O)ₐ(C₂H₄O)_{b}H

wherein b is from 2 to 150, and a is from 15 to 70. Generally speaking, block copolymers of ethylene oxide and propylene oxide meeting the above descriptions are available from BASF sold under the trademark “Pluronic and Lutrol F Block Copolymers”. For specifics of such polymers in detail, see BASF Corporation Technical Data Sheets on Pluronic polyols, copyright 1992. Using the poloxamer coding labels of BASF, suitable poloxamers for use in the invention include, but are not limited to:
Pluronic/Lutrol F 44 (poloxamer 124)
Pluronic/Lutrol F 68 (poloxamer 188)
Pluronic/Lutrol F 87 (poloxamer 237)
Pluronic/Lutrol F 108 (poloxamer 338)
Pluronic/Lutrol F 127 (poloxamer 407)

Polyoxamine tetrafunctional block copolymers, comprising four POE/POP blocks joined together by a central ethylenediamine bridge can also be employed to stabilize and protect the polystyrene surfaces in a closely analogous manner, the goal generally being to produce neutral or minimally-charged hydrophilic particles that are poorly recognized by Kupffer cells in the liver.

It has also been shown that surface modifications with poloxamers and poloxamines before intravenous injection is not always strictly necessary for making nanoparticles long-circulatory. Intravenously injected uncoated 60 nm polystyrene nanoparticles (which are susceptible to phagocytosis by Kupffer cells) were converted to long-circulating entities in rats that received a bolus intravenous dose of either poloxamer-407 or poloxamine-908, 1 to 3 h earlier (Moghimi, 1997, 1999.) It can be argued that the altered biodistribution profile of nanoparticles is the result of cell-surface modification by the administered copolymers. For instance, block copolymers could adhere to cell membrane hydrophobic domains via their hydrophobic center block or act as an effective membrane-spanning entity (Watrous-Peltier et al., 1992). The extracellular steric constraints resulting from hydrophilic POE tails of copolymers will then prevent the interaction between an approaching particle and the cell. Interestingly, this is apparently not the primary mechanism. Instead, nanoparticles have been shown to acquire a coating of copolymer and/or copolymer-protein complexes in the blood (Moghimi, 1997); this event explains their phagocytic resistance.

Microsphere surfaces in the present invention can also be coated with linear polyethylene glycol (PEG) to evade rapid blood clearance. The polymer backbone is essentially chemically inert, and the terminal primary hydroxyl groups are available for derivatization. Usually, the hydroxyl groups are first activated and then reacted with the chosen surface group; PEG activation and functionalization methods have been exhaustively reviewed (Zalipsky, 1995; Monfardini and Veronese, 1998). Surface modification of nanoparticles with PEG and its derivatives can be performed by adsorption, incorporation during the production of nanoparticles, or by covalent attachment to the surface of particles. Examples of currently available PEG conjugates for nanoparticle surface engineering includes PEG-R type copolymers, where R is PLA (Stolnik et al., 1994; Bazile et al., 1995), PLGA (Gref et al., 1994), and poly-ε-caprolactone (Shin et al., 1998; Kim et al., 1998) with appropriate molecular weights. The molecular weight of the PEG segment varies between 2000 and 5000, which is necessary to suppress protein adsorption.

Thus, the present microspheres are polymeric microspheres formulated to circulate within the body after injection (although inhalation may be another effective route of administration), and to be observable within the unperturbed living animal using optical equipment capable of exciting NIR dyes in the microspheres and of collecting, displaying, and analyzing the fluorescent light emitted from the particles. Controlled variables considered in preparing the imaging formulation include the absolute size and size distribution of the polystyrene particles, degree of chemical crosslinking of constituent polymer chains, optical properties of the dye or dyes used for impregnation, degree of dye loading, surface properties of the particles, state of aggregation of the native dyed particles, and types and amount of additional components (buffers, salts, surfactants, copolymers, etc.) present in the colloidal microsphere particle suspension comprising the injectable contrast imaging agent.

### Preparing the Fluorescent Microsphere

Fluorescent dyes have been incorporated into uniform microspheres in a variety of ways, for example by copolymerization of the fluorescent dye into the microspheres during manufacture (U.S. Pat. No. 4,609,689 to Schwartz et al. (1975), U.S. Pat. No. 4,326,008 to Rembaum (1982)); by entrapment of the fluorescent dye into the microspheres during the polymerization process; or by non-covalent incorporation of the fluorescent dye into previously prepared microspheres (U.S. Pat. No. 5,326,692). Each of these methods has previously been used to produce microparticles that are internally stained essentially throughout the interior of the particle.

The two basic means of preparing the microspheres of the invention are as follows: 1) bath dying of unstained or selectively stained microspheres; 2) Copolymerization of a fluorescent or non-fluorescent monomer onto the surface of an unstained or selectively stained microsphere. The above two techniques, when used alone or in combination, produce a variety of staining patterns within the subject microspheres.

### Fluorescent Dyes

Where the fluorescent microspheres of the invention are prepared by bath dying a pre-formed and unstained microsphere, the microspheres are typically stained using electrically neutral dyes that are generally hydrophobic. Where the microspheres of the invention are prepared by copolymerization of a fluorescent monomer with a nonfluorescent monomer (or monomers) the dye is required to have a functional group that will participate in the polymerization reaction so as to become covalently incorporated in the microsphere. These functional groups include but are not limited to fluorescent derivatives of styrenes and divinyl benzenes; acrylate and methacrylate acids, esters, amides and nitriles; vinyl and vinylidene halides, esters and ethers; alkenes, including ethylene, propylene, butadiene and isoprene; epoxides and isocyanates. In the case of fluorescent monomers while it is preferable that the dye be electrically neutral, it is not strictly essential.

The dye or dyes selected for incorporation into the microparticles are typically selected based upon the desired excitation and emission spectral properties that are readily determined by conventional means. The spectral properties of the fluorescent dyes should be determined in the polymeric materials in which they will be used. The excitation peak(s) of a dye can be approximately determined by recording an absorption spectrum on an absorption spectrophotometer or, more exactly, by running a fluorescent excitation spectrum using a scanning fluorescence spectrophotometer. The emission peak of the dye may also be determined using a fluorescence spectrophotometer to get an emission spectrum using a scanning fluorometer. The quantum yield of a candidate dye is typically determined by measuring with a fluorometer the total fluorescence emission of the dye in the desired polymer matrix, along with that of a reference dye with known absorbance at the excitation wavelength. The extinction coefficient is typically determined for a free dye in solution by using a spectrophotometer to measure absorbance of a solution with a gravimetrically determined concentration and calculating the extinction coefficient based on the Beer-Lambert law.

Once the spectral characteristics of a dye are determined in polymeric materials, as described above, those characteristics can be used to select the optimal dye or dye combination for a given application, taking into account the excitation source to be used, the available detection system, and the environment in which the materials will be used. Dyes useful for the invention generally have a quantum yield of greater than about 0.2 in the microsphere, preferably greater than about 0.5, as well as an extinction coefficient of greater than about 20,000 cm⁻¹M⁻¹, preferably greater than about 50,000 cm⁻¹M⁻¹. Dyes with lower quantum yields or lower extinction coefficients may be useful provided that sufficient concentrations can be incorporated within the microsphere so as to yield detectable fluorescent rings and/or disks.

Dyes that absorb light at the wavelengths of the principal excitation sources used in microscopy, and in particular those utilized for confocal laser scanning microscopy, are of particular importance for preparation of the fluorescent microspheres of the invention. These preferred absorbance wavelengths include those corresponding to the emission of the argon-ion laser (especially 350-360 nm, 454 nm, 488 nm and 514 nm), the krypton-ion laser (especially 568 nm and 647 nm), helium-neon lasers (especially 543 nm, 592 nm and 633 nm), mercury arc lamps (especially near 365 nm and 545 nm) and various other excitation sources, including laser diodes, frequency-doubled lasers and other light sources.

The polymeric microspheres of the present invention that are efficiently excited at wavelengths from the ultraviolet region to about 480 nm can be prepared using a wide variety of electrically neutral dyes. Many of these are known and widely used as laser dyes such as those commercially available from Lambda Electronics (Melville, N.Y.) and by Exciton. Useful dyes include, but are not limited to a pyrene, an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a carbocyanine (including any corresponding compounds in U.S. Ser. Nos. 09/557,275; 09/968,401 and 09/969,853 and U.S. Pat. Nos. 6,403,807; 6,348,599; 5,486,616; 5,268,486; 5,569,587; 5,569,766; 5,627,027; 6,048,982 and 6,664,047), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. Nos. 6,162,931; 6,130,101; 6,229,055; 6,339,392; 5,451,343 and U.S. Ser. No. 09/922,333), an oxazine or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including an corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, oxazines include resorufins (including any corresponding compounds disclosed in 5,242,805), aminooxazinones, diaminooxazines, and their benzo-substituted analogs.

Where the dye is a xanthene, the dye is optionally a fluorescein, a rhodol (including any corresponding compounds disclosed in U.S. Pat. Nos. 5,227,487 and 5,442,045), a rosamine or a rhodamine (including any corresponding compounds in U.S. Pat. Nos. 5,798,276; 5,846,737; 5,847,162; 6,017,712; 6,025,505; 6,080,852; 6,716,979; 6,562,632). As used herein, fluorescein includes benzo- or dibenzofluoresceins, seminaphthofluoresceins, or naphthofluoresceins. Similarly, as used herein rhodol includes seminaphthorhodafluors (including any corresponding compounds disclosed in U.S. Pat. No. 4,945,171).

Preferred dyes of the invention include rhodol, fluorescein, rhodamine, dansyl, benzofuran, indole, cyanine, quinazolinone, pyrene, naphthalene, coumarin, oxazine, oxazole, benzofuran, indole, a benzazole and borapolyazaindacene. In one embodiment benzofuran and benzazole form a fused reporter molecule with either benzo ring the chelating moiety. In another aspect, the reporter molecules xanthene, dansyl, benzofuran, indole, cyanine, quinazolinone, pyrene, naphthalene, coumarin, oxazine, indole, and borapolyazaindacene are independently attached to the chelating moiety by a linker.

In an exemplary embodiment, the dye contains one or more aromatic or heteroaromatic rings, that are optionally substituted one or more times by a variety of substituents, including without limitation, hydrogen, amino, substituted amino, halogen, nitro, sulfo, cyano, alkyl, substituted alkyl, perfluoroalkyl, alkoxy, alkenyl, alkynyl, cycloalkyl, arylalkyl, acyl, aryl or heteroaryl ring system, substituted aryl, benzo, solid support, reactive group, carrier molecule, lipophilic group, or other substituents typically present on chromophores or fluorophores known in the art.

In one aspect, the dyes are independently substituted by substituents selected from the group consisting of hydrogen, halogen, amino, substituted amino, alkyl, substituted alkyl, aryl, substituted aryl, heteroaryl, substituted heteroaryl, alkoxy, sulfo, reactive group, solid support, lipophilic group, and carrier molecule. In another embodiment, the xanthene dyes of this invention comprise both compounds substituted and unsubstituted on the carbon atom of the central ring of the xanthene by substituents typically found in the xanthene-based dyes such as phenyl and substituted-phenyl moieties.

In an exemplary embodiment, the dye has an absorption maximum beyond 480 nm. In a particularly useful embodiment, the dye absorbs at or near 488 nm to 514 nm (particularly suitable for excitation by the output of the argon-ion laser excitation source) or near 546 nm (particularly suitable for excitation by a mercury arc lamp). As is the case for many dyes, they can also function as both chromophores and fluorophores, resulting in compounds that simultaneously act both as colorimetric and fluorescent labels for heavy metal ions. Thus, the described fluorescent dyes are also the preferred chromophores of the present invention.

Enzymes are desirable reporter molecules because amplification of the detectable signal can be obtained, resulting in increased assay sensitivity. The enzyme itself does not produce a detectable response, but functions to break down a substrate when it is contacted by an appropriate substrate such that the converted substrate produces a fluorescent, colorimetric or luminescent signal. Enzymes amplify the detectable signal because one enzyme can result in multiple substrate molecules being converted to a detectable signal. This is advantageous where there is a low quantity of microspheres present in the sample or a dye does not exist that will give comparable or stronger signal than the enzyme. The enzyme substrate is selected to yield the preferred measurable product, e.g. color, fluorescence or chemiluminescence. Such substrates are extensively used in the art, many of which are described in the INVITROGEN, THE HANDBOOK, supra.

A preferred colorimetric or fluorogenic substrate and enzyme combination uses oxidoreductases such as horseradish peroxidase (HRP) and a substrate such as 3,3′-diaminobenzidine (DAB) or 3-amino-9-ethylcarbazole (AEC), which yield a distinguishing color (brown and red, respectively). Other colorimetric oxidoreductase substrates that yield detectable products include, but are not limited to: 2,2-azino-bis(3-ethylbenzothiazoline-6-sulfonic acid) (ABTS), o-phenylenediamine (OPD), 3,3′,5,5′-tetramethylbenzidine (TMB), o-dianisidine, 5-aminosalicylic acid and 4-chloro-1-naphthol. Fluorogenic substrates include, but are not limited to, homovanillic acid or 4-hydroxy-3-methoxyphenylacetic acid, reduced phenoxazines and reduced benzothiazines, including Amplex® Red reagent and its variants (U.S. Pat. No. 4,384,042) and reduced dihydroxanthenes, including dihydrofluoresceins (U.S. Pat. No. 6,162,931) and dihydrorhodamines, including dihydrorhodamine 123. Peroxidase substrates that are tyramides (U.S. Pat. Nos. 5,196,306; 5,583,001 and 5,731,158) represent a unique class of peroxidase substrates in that they can be intrinsically detectable before action of the enzyme but are “fixed in place” by the action of a peroxidase in the process described as tyramide signal amplification (TSA). These substrates are extensively utilized to label targets in samples that are cells, tissues or arrays for their subsequent detection by microscopy, flow cytometry, optical scanning and fluorometry.

Another preferred colorimetric (and in some cases fluorogenic) substrate and enzyme combination uses a phosphatase enzyme such as an acid phosphatase or a recombinant version of such a phosphatase in combination with a colorimetric substrate such as 5-bromo-4-chloro-3-indolyl phosphate (BCIP), 6-chloro-3-indolyl phosphate, 5-bromo-6-chloro-3-indolyl phosphate, p-nitrophenyl phosphate, or o-nitrophenyl phosphate or with a fluorogenic substrate such as 4-methylumbelliferyl phosphate, 6,8-difluoro-7-hydroxy-4-methylcoumarinyl phosphate (DiFMUP, U.S. Pat. No. 5,830,912), fluorescein diphosphate, 3-O-methylfluorescein phosphate, resorufin phosphate, 9H-(1,3-dichloro-9,9-dimethylacridin-2-one-7-yl) phosphate (DDAO phosphate), or ELF 97, ELF 39 or related phosphates (U.S. Pat. Nos. 5,316,906 and 5,443,986).

Glycosidases, in particular β-galactosidase, β-glucuronidase and β-glucosidase, are additional suitable enzymes. Appropriate colorimetric substrates include, but are not limited to, 5-bromo-4-chloro-3-indolyl β-D-galactopyranoside (X-gal) and similar indolyl galactosides, glucosides, and glucuronides, o-nitrophenyl β-D-galactopyranoside (ONPG) and p-nitrophenyl β-D-galactopyranoside. Preferred fluorogenic substrates include resorufin β-D-galactopyranoside, fluorescein digalactoside (FDG), fluorescein diglucuronide and their structural variants (U.S. Pat. Nos. 5,208,148; 5,242,805; 5,362,628; 5,576,424 and 5,773,236), 4-methylumbelliferyl β-D-galactopyranoside, carboxyumbelliferyl β-D-galactopyranoside and fluorinated coumarin β-D-galactopyranosides (U.S. Pat. No. 5,830,912).

Additional enzymes include, but are not limited to, hydrolases such as cholinesterases and peptidases, oxidases such as glucose oxidase and cytochrome oxidases and reductases for which suitable substrates are known.

Enzymes and their appropriate substrates that produce chemiluminescence are preferred for some assays. These include, but are not limited to, natural and recombinant forms of luciferases and aequorins. Chemiluminescence-producing substrates for phosphatases, glycosidases and oxidases such as those containing stable dioxetanes, luminol, isoluminol and acridinium esters are additionally useful. Several chemiluminescent substrates for phosphatase enzymes are known, including the BOLD APB chemiluminescent substrate (Molecular Probes, Inc.).

Fluorescent proteins also find use in the microspheres of the present invention. Examples of fluorescent proteins include green fluorescent protein (GFP) and the phycobiliproteins and the derivatives thereof. The fluorescent proteins, especially phycobiliproteins, are particularly useful for creating tandem dye-reporter molecules. These tandem dyes comprise a fluorescent protein and a fluorophore for the purposes of obtaining a larger Stokes shift, wherein the emission spectra are farther shifted from the wavelength of the fluorescent protein's absorption spectra. This property is particularly advantageous in the present in vivo applications where the background from other particles is large. For this to work, the fluorescent protein and fluorophore function as an energy transfer pair wherein the fluorescent protein emits at the wavelength that the acceptor fluorophore absorbs and the fluorophore then emits at a wavelength farther from the fluorescent proteins than could have been obtained with only the fluorescent protein. Alternatively, the fluorophore functions as the energy donor and the fluorescent protein is the energy acceptor. Particularly useful fluorescent proteins are the phycobiliproteins disclosed in U.S. Pat. Nos. 4,520,110; 4,859,582; 5,055,556 and the fluorophore bilin protein combinations disclosed in U.S. Pat. No. 4,542,104.

Alternatively, two or more fluorophore dyes can function as an energy transfer pair (tandem dye) wherein one fluorophore is a donor dye and the other is the acceptor dye including any dye compounds disclosed in U.S. Pat. Nos. 6,358,684; 5,863,727; 6,372,445; 6,221,606; 6,008,379; 5,945,526; 5,863,727; 5,800,996; 6,335,440; 6,008,373; 6,184,379; 6,140,494 and 5,656,554. This provides the potential for a large stokes shift where the excitation and emission spectra can be very different.

Exemplary semiconductor nanocrystals and methods of there preparation are described in U.S. Pat. Nos. 6,207,299, 6,322,901 and 6,576,291, and in the publication “Alternative Routes toward High Quality CdSe Nanocrystals,” (Qu et al., Nano Lett., 1(6):333-337 (2001)). The use of alloyed or mixed shells has been described in U.S. Pat. No. 6,815,064. The use of a promoter to make nanocrystal cores has been described in U.S. Patent Publication No. 2003/0097976 (published May 29, 2003). Surface modification methods in which mixed hydrophobic/hydrophilic polymer transfer agents are bound to the surface of the nanocrystals are suggested in U.S. Pat. No. 6,649,139.

Methods for making nanocrystal impregnated microspheres are also well-known in the art. The preparation of nanocrystal impregnated microspheres has been the subject of many patents and publications, the following are several examples. U.S. Pat. No. 6,479,146 describes methods using electrostatic self-assembly of nanocomposite multilayers on decomposable colloidal templates. International Publication No. WO 00/77281 (published Dec. 21, 2000) described encapsulation of crystals via multilayer coatings. International Publication No. WO 01/51196 (published Jul. 19, 2001) described the templating of solid particles using polymer multilayers. International Publication No. WO 99/47252 (published Sep. 23, 1999) described the use of layer-wise polyelectrolyte self-assembly to prepare nanocapsules and microcapsules. U.S. Pat. Nos. 6,548,171 B1 and 6,680,211 B2 describe microspheres with embedded fluorescent nanocrystals. Finally, polyelectrolyte multilayer films were modeled by Park et al., Langmuir 18: 9600-9604 (2002).

### Bath Dying.

Bath dying refers to the absorption of a dye or dyes into the microsphere directly from solvent. Somewhat hydrophobic fluorescent dyes, being freely soluble in organic solvents and very sparingly soluble in water, are readily introduced by solvent-based addition of the dye to previously formed microspheres.

Bath dying has previously been used to produce fluorescent (and colored) microspheres without regard to producing a specific spherical staining pattern of staining Bath-staining techniques as described in the invention are required to prevent substantial penetration of the dye into the microspheres in order to produce a distinct spherical zone near the surface of the microsphere. In particular, a variety of parameters must be carefully controlled in order for distinct shallow staining to occur, including solvent polarity, the complete absence of water in the staining solution, the physical characteristics of the dyes utilized, the composition of the microsphere, and the staining duration.

The solvent combination utilized for the staining solution should swell polymeric matrix of the microspheres enough so that staining is controlled, but not enough to permanently damage the microsphere itself, or to allow excessive amounts of fluorescent dye already present to ‘bleed’ from the microsphere. The degree of swelling of the microspheres is typically manipulated by controlling the amount of chlorinated organic solvent present in the staining solution. Chlorinated solvents include, among others, methylene chloride and chloroform, preferably methylene chloride. While for uniform staining of the microspheres, the staining solution contains 25% or more chlorinated solvent, preferably greater than 30%, but for applying shallow staining the staining solution should contain less than 25% chlorinated solvent, preferably less than 20%.

Shallow staining occurs under anhydrous conditions. The presence of water during the shallow staining procedure typically causes precipitation of the fluorescent dye, and agglutination of the microspheres. Compensation for the presence of water by the addition of more chlorinated solvent results in excessive swelling of the microspheres resulting in a complete loss of shallow staining, and permanent damage to the microsphere. All traces of water must be carefully excluded from the staining solution in order to achieve shallow staining

Fluorescent dyes used for shallow staining must be selected to have hydrophobicities and steric properties consistent with the staining requirements. In particular, the dyes must be largely nonpolar (electrically neutral), and possess a structural geometry consistent with intercalation into the polymeric matrix. Extremely large or excessively bulky dyes will be prevented from diffusing into the microsphere interior, while dyes that are not sufficiently hydrophobic will fail to be well-retained after dye preparation, in both cases resulting in inferior shallow staining

Similarly, a dye selected as a uniform stain for the interior of the microsphere must be both hydrophobic and sterically bulky enough to resist diffusion out of the microsphere during the brief exposure to solvents while the shallow staining is being performed.

In principle, the microspheres must remain in contact with the staining solution long enough for the suspension to become essentially homogeneous, and for the desired degree of staining to occur. While precisely defined staining times are not needed, staining times of less than 10 minutes are typically utilized, more typically less than 5 minutes, and preferably the microspheres are kept in the staining solution for about 1 minute.

Bath dying can utilize a single dye or, multiple dyes may be used to produce spherical zones that are partially or fully coincident. Multiple dyes are also utilized to produce extensive energy transfer within the stained region, mixing the dyes in the dying solution according to ratios selected to give desired combinations of spectral properties.

Polymerization onto an Existing Core

It is common for unstained microspheres that have a uniform diameter to be prepared through multiple polymerization reactions, each successive step adding new coating to the surface of the microsphere (Bangs, UNIFORM LATEX PARTICLES, 1984, Seragen, Inc.). Modification of this method of microsphere preparation can be used to produce microspheres with one or more fluorescent spherical zones. This method is particularly useful for producing one or more discrete fluorescent zones that are well within the microsphere, for example by selection of either fluorescent or non-fluorescent monomers for additional polymerization steps.

Preparing microspheres with nonfluorescent cores is analogous to preparing those with fluorescent cores except that the initial microsphere is essentially nonfluorescent or already contains a fluorescent spherical zone. When a nonfluorescent core is coated with a fluorescent monomer (or monomers) then a similar pattern of ring staining at or near the surface is observed as is produced using bath dying. Utilizing copolymerization of a fluorescent monomer on a nonfluorescent core has the advantage that the resulting spherical fluorescent zones do not diffuse either deeper into (or out of) the microsphere.

### Combined Techniques

The two techniques may be combined to produce exceptionally powerful methods for producing a desired staining pattern in the subject microsphere. As bath dying is typically utilized to produce a shallowly stained microsphere, a microsphere may be bath dyed to produce a narrow spherical zone of fluorescent labeling, followed by copolymerization of additional fluorescent or nonfluorescent monomer to produce an internal fluorescent spherical zone. The resulting microsphere can then be subjected to bath dying again to produce additional distinct shallow staining

In an additional embodiment, polymeric cores that are relatively impermeant to dye absorption from solvent are coated in a subsequent polymerization step with a second layer that is more receptive to bath dying. The core of such a microsphere may be selected so as to retard or prevent subsequent migration of dye further into the interior of the microsphere. In yet another embodiment of the invention, the core of the microsphere is paramagnetic and fluorescent or nonfluorescent and also contains a shallow ring stain.

In each embodiment of the invention, the microparticles utilized for the invention can be prepared or purchased with a variety of surface properties, with functional groups including, but not limited to sulfate, phosphate, hydroxyl, carboxyl, ester, amide, amidine, amine, sulfhydryl, chloromethyl and aldehyde, so long as the surface is hydrophobic. If required, some of these groups may be activated for coupling to members of specific binding pairs or other surfaces. The surface groups can also be selected so as to give the particles desired physical characteristics, such as varying degrees of hydrophobicity, or to provide another means of attachment for a member of a specific binding pair.

### Illumination/Excitation

The sample containing a microsphere may be illuminated with a wavelength of light selected to give a detectable optical response, and observed with a means for detecting the optical response. By optical response is meant any detectable colorimetric or luminescent property of the complex. Typically, the optical response is related to the excitation or emission properties of the fluorescent dye.

For example, the sample may be excited by a light source capable of producing light at or near the wavelength of maximum absorption of the fluorescent dye, such as an ultraviolet or visible lamp, an arc lamp, a laser, or even sunlight. The optical response is optionally detected by visual inspection, or by use of any of the following devices: CCD camera, video camera, photographic film, laser-scanning devices, fluorometers, photodiodes, quantum counters, epifluorescence microscopes, scanning microscopes, flow cytometers, fluorescence microplate readers, or by means for amplifying the signal such as photomultiplier tubes. Where the sample is examined using a flow cytometer, examination of the sample optionally includes sorting portions of the sample according to their fluorescence response.

The present hydrophobic microspheres can be used in any method known in the art for optical contrast agents. Among the applications for passively accumulating optical contrast agents are sentinel lymph node tracing, endoscopic and colonoscopic or cytoscopic procedures, and cancer detection. In addition to the obvious case of examining skin lesions, colonoscopy, bronchoscopy, upper gastrointestinal endoscopy, and laparoscopy, which all provide surface illumination of deeper epithelial tissue at risk for neoplasia. In humans, fluorescence colonoscopy as well as fluorescent evaluation of other tissues such as bladder, larynx, esophagus and lung have been performed. Rat and mouse colonoscopy or cytoscopy have also been reported in which one may visually inspect tissues in full color (white light illumination) while observing in a NIR channel another independent parameter, such as vascular leakiness or protease activity. In another application, the contrast agents can be used to guide tissue resection during surgical removal of tumors by providing optical contrast between diseased and non-diseased tissue.

After the present fluorescent microspheres have accumulated at the site of disease or injury in the body the contrast agents are visualized using optical imaging instrumentation. A number of optical imaging approaches are known to those skilled in the art, including, but not limited to the method taught in U.S. Pat. No. 5,422,730. These techniques rely on fluorescence, absorption, reflectance, or bioluminescence as the source of contrast, while imaging systems can be based on diffuse optical tomography, surface-weighted imaging (reflectance diffuse tomography), phase-array detection, confocal imaging, multiphoton imaging, or microscopic imaging with intravital microscopy. With the exception of near-infrared fluorescence imaging and superficial confocal and two-photon imaging, these techniques currently are primarily limited to experimental imaging in small animals.

Near-infrared fluorescence imaging relies on light with a defined bandwidth as a source of photons that encounter a fluorescent molecule (optical contrast agent), which emits a signal with different spectral characteristics that can be resolved with an emission filter and captured with a high-sensitivity charge-coupled-device camera.

Fluorescence-based optical imaging instrumentation can be based on planar continuous wave reflectance or time-domain-based phenomena. Time-domain based optical imaging can quantitatively recover depth, volume, concentration, and fluorescent lifetime of different light emitting molecular probes using both photon temporal distribution and intensity data. Imaging instrumentation based on diffuse reflectance is available from various instrument makers, including Cambridge Research and Instrumentation, Inc. (Woburn, Mass.), and VisEn Medical (Woburn, Mass.). Time-domain-based imaging instrumentation is available from GE Healthcare Technologies (Waukesha, Wis.).

The wavelengths of the excitation and emission bands of the microspheres vary with reporter molecule composition to encompass a wide range of illumination and detection bands. This allows the selection of individual reporter molecules for use with a specific excitation source or detection filter. In particular, present reporter molecules/dyes can be selected that possess excellent correspondence of their excitation band with the 488 nm band of the commonly used argon laser or emission bands which are coincident with preexisting filters.

The foregoing methods having been described, it is understood that the many and varied compositions of the present invention can be utilized with the many methods.

### Instrument Evaluation and Correction

The microspheres of the present invention possess utility for improving the performance of any instrument capable of three-dimensional spatial analysis. While confocal laser scanning microscopy is the apparently the most common instrument used for three-dimensional analysis, any other method of microscopy that yields three-dimensional information about a specimen, such as wide-field microscopy coupled with image deconvolution, can be evaluated and/or calibrated using the microspheres of the invention.

The microspheres essentially function as microscopic three-dimensional gauges. Microspheres are isotropic, i.e. their staining pattern does not depend on the orientation of the microsphere with respect to the illumination utilized. Upon examination of the microsphere, as processed by the instrument to be evaluated, any deviation of the staining pattern from the known characteristics of the microsphere indicates inaccuracy in either the physical optics of the instrument, the data acquisition parameters, or in post-acquisition data analysis.

For evaluating and calibrating an instrument, the instrument is first used to generate a three-dimensional representation of one or more microspheres of the present invention. The three-dimensional representation can be, for example, an actual optical image, and electronic image, a set of optical cross-sections, or a three-dimensional data array. The three-dimensional representation is then compared with the expected three-dimensional representation, which is based on knowledge of the actual physical and spectral characteristics of the microspheres. In comparing the experimental data with the expected result, the performance of specific operating parameters of the instrument can be evaluated. Once the instrument has been evaluated, the operating parameters of the instrument are then adjusted so to make the three-dimensional representation more accurate with respect to the known physical and spectral characteristics of the microsphere (e.g., restoring the circularity of the image, or correcting a lack of superimposition).

In one aspect of the invention, the microspheres are used to evaluate, align, and calibrate the optical elements of the instrument, from the objective lens to the detector. By optical elements is meant both the excitation and collection optics. Elements of the optical path subject to adjustment or evaluation include, for example, excitation sources, lenses, relay mirrors, scanning mirrors, dichroics, beamsplitters, filter wheels and filter blocks. Examples of the types of evaluation and calibration possible include, evaluation of the objective lens to aid in appropriate lens selection, evaluation of the flatness of the optical field (or spherical aberration), evaluation of the chromatic registration in the optical field, i.e. chromatic aberration in the x-y or x-z axis, and aiding in identifying the need for correction in the ultraviolet region or other wavelengths.

It has traditionally been especially difficult for users of confocal laser scanning instruments to detect and correct for chromatic aberration along the z-axis. Certain microspheres of the invention are particularly useful in this regard. There microspheres have at least one fluorescent spherical zone that contains multiple dyes, where each dye has a different emission maximum and gives a distinct ring. Such a microsphere should yield coincident fluorescent rings in the x-y plane, at every position along the z-axis. The appearance of multiple nonsuperimposed rings in different fluorescence channels indicates chromatic aberration or misalignment of optical components and adjustments can therefore be made to restore coincidence of the rings.

In another aspect of the invention, the microspheres of the invention are used in conjunction with evaluating data acquisition parameters. For example, evaluation of image resolution, image intensity, magnification and detector sensitivity allows for acquisition parameters to be adjusted to maximize image accuracy.

In another aspect of the invention, the microspheres of the invention are utilized in conjunction with post-acquisition data analysis. For example, the microspheres of the invention are useful for facilitating image deconvolution using wide-field microscopy. Additionally, the microspheres possess utility for facilitating image correction and image reconstruction, with respect to making the x, y and z axes coincident in each emission channel. Alternatively, the microspheres are used to identify inaccuracies in volume reconstruction calculations, or to correct for errors in post-acquisition color representation.

Similarly, the microspheres of the present invention facilitate the determination of both the magnitude and anisotropy of chromatic aberration or spherical aberration, and can facilitate either physical corrections, corrections to the data acquisition parameters, or corrections to the post-acquisition data analysis to compensate for such chromatic aberration or spherical aberration.

In general, the microspheres of the invention are used to detect equipment malfunction or failure, to verify that collected data accurately represents the specimen of interest, and in general to “troubleshoot” every aspect of the instrument being utilized.

### Multiplexing:

An advantage of fluorescent microspheres of the present invention is the potential for multi-color applications (multiplexing). Ideal fluorophores for multi-color applications have comparable fluorescence intensity, narrow spectral bandwidth and maximal spectral separation (West et al., 2001). Anywhere from 2 to 20 differently labeled microspheres can be used. In one embodiment the microspheres are encoded with different dyes or combinations of dyes. In some embodiments, the microspheres are encoded with quantum dot semiconductor nanocrystals.

The microspheres can either be in population having the same size or different sizes. In one embodiment two different populations of microspheres having distinct diameters (sizes) are labeled with different colors. Studies involving flow rates for different sized particles then become possible. In one aspect, different sized particles are co-administered and different sized passageways can be concomitantly monitored, such as capillaries vs. arteries and veins. Another particular application involves monitoring of dilation, constriction or occlusion of the vasculature through measurement with multiple color/sized microspheres, wherein only microspheres smaller than the diameter of the artery, vein or capillary can enter. The present hydrophobic microspheres are particularly advantageous because of there ability to travel without sticking to biological particles, which would otherwise cause blockage of particular channels.

### Applications:

The method of the present invention is particularly useful in the diagnosis of particular disease states, as well as a method for determining efficacy of a particular therapy or surgery. For diagnosis, aberrations in blood flow, such as increased turbulence, decreased or increased blood flow velocity, or total blockage can be clear indicators of disease states and the level or severity thereof.

In a particular example, the blood flow measurements are used to identify vascular occlusion or infarctions due to clots, build up of adhesion molecules, inflammation, fatty acid build-up (e.g. cholesterol), plaques, or hardening of the arteries. The blood flow may either be reduced, blocked or the flow may be more turbulent due to any of these factors. This can provide for the diagnosis and/or prevention of heart attacks, ischemia, stroke, peripheral vascular disease, carotid artery disease, renal artery disease, or abnormal aortic aneurysm.

In another embodiment, the blood flow measurements are used to identify arrhythmias. Factors such as turbulence or modulation of blood flow velocity may be indicators of arrhythmias or cardiomyopathies.

In another embodiment, the blood flow measurements are used to identify tumors or cancer. Factors such as turbulence or modulation of blood flow velocity may be indicators of cancer and/or tumors.

In another embodiment, the blood flow measurements are used to identify infection, such as from bacteria or a virus. Factors such as turbulence or modulation of blood flow velocity are indicators of infection.

Particular antigens present in the blood stream that may indicate a disease state are identified by binding of the microspheres functionalized with an antibody or receptor capable of binding the antigen. Subsequent reduction or modulation of microsphere mobility indicates binding of the antigen and presence of the disease state.

In another embodiment, the blood flow measurements are used to identify the efficacy of a particular line of treatment, such as a drug. Particularly, the blood flow measurements can be taken before and after administration of the drug, wherein return to a healthy or normal blood flow indicates efficacy of the drug. In a particular aspect, the drug is for reduction or prevention of arterial plaque or clot formation and the reduction or elimination of turbulence in areas suspected of containing plaques or clots indicates efficacy of the drug. Preferably, the drug is a statin.

In another embodiment, the blood flow measurements are performed on a patient undergoing surgery or post surgery to determine the effectiveness of the surgery and/or any post-operative conditions. The surgery can be for the heart, such as bypass surgery, or any other organ or tissue associated with the vasculature. Additionally, the surgery may involve an implant, such as a stent or pacemaker, wherein the blood flow and turbulence can be monitored around the insertion of the implant.

The blood flow measurements may also be localized to a particular organ, tissue, or region of the body. Preferred locations include, the vascular system, the heart, aorta, arteries, veins, liver, kidneys, spleen, eyes, retina, skin, breasts, testes, colon, prostate, lungs, lymphatic system, brain, or spinal cord.

### Specific Binding Pair Members

In one aspect of the invention, the surface of the microsphere of the invention is further modified to be covalently or noncovalently attached to a member of a specific binding pair (i.e. another agent). Each specific binding pair member has an area on the surface or in a cavity that specifically binds to and is complementary with a particular spatial and polar organization of its complementary specific binding pair member. A specific binding pair member can be a ligand or a receptor. As used in this document, the term ligand means any organic compound for which a receptor naturally exists or can be prepared. A receptor is any compound or composition capable of recognizing a spatial or polar organization of a molecule, e.g. epitopic or determinant site. Ligands for which naturally occurring receptors exist include natural and synthetic peptides and proteins, including avidin and streptavidin, antibodies, enzymes, and hormones; nucleotides and natural or synthetic oligonucleotides, including primers for RNA and single- and double-stranded DNA; polysaccharides and carbohydrates. Representative specific binding pairs are shown in Table 1.

TABLE 1
Representative Specific Binding Pairs

| | |
|---|---|
| antigen | antibody |
| biotin | avidin (or streptavidin) |
| IgG* | protein A or protein G |
| drug receptor | drug |
| toxin receptor | toxin |
| carbohydrate | lectin |
| peptide receptor | peptide |
| protein receptor | protein |
| carbohydrate receptor | carbohydrate |
| DNA (RNA) | aDNA (aRNA)^{†} |
| enzyme | substrate |

*IgG is an immunoglobulin
^{†}aDNA and aRNA are the antisense (complementary) strands used for hybridization

In one aspect of the invention, the specific binding pair member is an antibody or antibody fragment, avidin or streptavidin. In this embodiment of the invention, the complementary binding pair member is typically a hapten, including drugs, an antigen or a biotin. Where the complementary binding pair member is a hapten, the hapten typically has a molecular weight less than 1000 daltons. In another aspect of the invention, the specific binding pair member is an oligonucleotide or nucleic acid polymer. Optionally, the complementary binding pair member is present in a cell, bacteria, virus or yeast cell such as an Fc receptor. Alternatively, the complementary member is immobilized on a solid or semi-solid surface, such as a polymer, polymeric membrane (such as polyvinylidene difluoride or nitrocellulose) or polymeric particle (such as an additional microsphere), a microchip array, or in a semi-solid matrix (such as an electrophoresis gel).

In a particular aspect of the invention the additional agent bound to the microsphere provides a means for identifying and binding particles in the blood of a subject. More particularly, the particles can be associated with a particular disease afflicting the subject. Examples include antigens associated with a virus, such as influenza, which includes hemagglutinin (HA) and neuraminidase (NA). Alternatively, the particles can be associated HIV or HCV infection. Furthermore, the particles may be associated with diseases such as cancer, wherein the over expression of particles, such as kinases, are detected. Alternatively, the disease may affect inflammatory processes indicated by molecules such as cytokines and TLR (toll-like receptors).

When bound to a particle, such as those described above, the net velocity of the microsphere-containing complex will be reduced. The severity of the disease is then inversely proportional with the velocity of the microspheres. Alternatively, where the disease causes a reduction in the particles of interest, such as the reduction in killer T-cells by HIV, the velocity of the microspheres will increase, and disease severity will be directly proportional with the increase in velocity of the microspheres (as compared with a control, e.g. a healthy patient). This technology will find many applications, including for use in rapidly identifying patients and/or birds afflicted with the avian flu.

It is also contemplated that a number of different microspheres, each having a different agent (i.e. binding pair) associated with it, and a separately detectable dye, can be used simultaneously such that a number of different diseases can be tested for at once. For example, a patient having a cancer associated with the over expression of a particular growth factor, such as EGF, can quickly be identified by the detection of microspheres having a receptor for that particular growth factor (among others). Accordingly, the patient can receive a treatment specific for that pathway, such as gefitinib (Iressa®).

Notwithstanding, in a particularly preferred embodiment the microspheres are unsubstituted (i.e. without any functionalization) such that they are free to move about in the blood stream unaffected or minimally affected by other particles therein.

### Kits

Suitable kits comprising hydrophobic fluorescent microspheres also form part of the present disclosure. Such kits can be prepared from readily available materials and reagents and can come in a variety of embodiments. The contents of the kit will depend on the design of the assay protocol or location of blood flow for detection or measurement. All kits will contain instructions, appropriate materials, and two or more of the presently disclosed fluorescent microspheres. Typically, instructions include a tangible expression describing the microsphere concentration or administration method, time periods for sample detection, temperatures, additives/buffer conditions and the like to allow the user to carry out any one of the methods or preparations described above. In one aspect, the kit is formulated to facilitate ease of handling and administration. This involves a pre-packed syringe or other vessel, such as a nebulizer or inhalation device, which is ready for administration to a subject.

Therefore, kits of the present invention comprise at least two microspheres of the present invention in an appropriate storage form, e.g. lyophilized or dissolved in an organic solvent, and instructions for preparing the microspheres to be used by the end user.

In one embodiment a kit for measuring blood flow in a subject comprises:

a plurality of microspheres, wherein the microspheres are impregnated with a dye having an excitation and emission spectrum compatible with in vivo or intravital imaging and further, wherein the microspheres have a hydrophobic outer surface;

packaging; and

written instructions on how to use the microspheres for the detection of blood flow.

In addition, the kits may contain appropriate controls (including a positive control), calibration standards, ample preparation reagents, an aqueous dilution buffer, an organic solvent, additional microspheres, binding pairs, an antibody or fragment thereof or a reference dye standard.

A detailed description of the invention having been provided above, the following examples are given for the purpose of illustrating the invention and shall not be construed as being a limitation on the scope of the invention or claims.

### EXAMPLES

Hydrophobic polystyrene-based microspheres are synthesized according to well-known polymerization methods using styrene monomers, an initiator, a buffer, and a surfactant. Chain initiation occurs when a sulfate free radical reacts with the double bond of a styrene monomer. The resulting styrene free radical reacts with additional molecules of styrene to produce large chains of polystyrene. Chain termination occurs when growing chains react to make a sulfate-terminated polymer chain. The polystyrene chains spontaneously coalesce in water to form spheres due to their hydrophobic nature. Once formed, the spheres are soaked with fluorescent dyes, which then become integrated within the internal matrix of the spheres.

Controlled variables considered in preparing the described fluorescent microspheres include the absolute size and size distribution of the particles, surface properties of the microspheres (amount of charge content), degree of chemical crosslinking of constituent polymer chains, optical properties of the dye or dyes used for impregnation, degree of dye loading related to emission intensity and possible internal quenching effect, state of aggregation of the regular and sterilized fluorescent particles, and types and amount of additional components (buffers, salts, surfactants, copolymers, etc.) present in the described fluorescent microspheres in this invention.

In the one example, the parameters of the microspheres are:
Particle size: from 100 nm up to 4 μm
Particle size distribution: ±4% diameter
Chemical crosslinking: none
Optical properties of dyes: four BODIPY dyes with excitation max at: 488 nm/510 nm, 545 nm/570 nm, 675 nm/700 nm and 715 nm/755 nm respectively and a tetraphenyl butadiene dye with ex/em at 350 nm/440 nm.
Degree of dye loading: obtained maximum emission intensity without quenching
Surface properties: hydrophobic, very low surface charge content: 1-6 μEq/gram,
Aggregation state: nanometer-sized microspheres are at non-settling colloidal suspension; more than 95% of the 1 μm to 4 μm sized microspheres are at mono-dispersed singlet state before and after sterilization process.

Batches of hydrophobic microspheres were synthesized having the following charge and size dimensions: 6 μEq/gm (100 nm), 3.1 μEq/gm (200 nm), 6.2 μEq/gm (500 nm), 2.8 μEq/gm (1 μm), 2.5 μEq/gm (2 μm) and 1 μEq/gm (4 μm).

These are compared with hydrophilic CML microspheres which were shown to have the following charge and size dimensions: 320 μEq/gm (100 nm), 88 μEq/gm (200 nm), 142 μEq/gm (500 nm), 176 μEq/gm (1 μm), 153 μEq/gm (2 μm) and 56.3 μEq/gm (4 μm).

### Example 1

### Preparation of 0.1 μm Microspheres with NIR Emission (715 ex/755 em)

The staining solution is prepared by adding 700 μL of the dye stock (BODIPY® 670/735 [difluoro(1-((3-(2-(5-hexyl) thienyl)-2H-isoindol-1-yl)methylene)-3-(2-(5-hexyl)thienyl)-1H-isoindolato-N¹,N²)boron] 10 mg/mL stock solution in CH₂Cl₂) to a 10 mL glass test tube, adding 300 μL of CH₂Cl₂, and mixing. 2 mL of Ethanol is then added and sonicated for 30 second to ensure complete mixing. The microspheres are loaded with dye by first adding 10 ml of a vortexed microsphere stock (0.11 μm sulfate polystyrene microspheres (0.1 μm), 8.1% solids, with surface charge content of 6 μEq/g (measured from conductometric titration)) to a 250 ml round bottom flask and then slowly adding 14 mL of methanol and stirring for 5 minutes. The staining solution is added dropwise to the stirred microsphere suspension and incubated for 30 minutes with continual stirring. The organic solvents are evaporated in a BUCHI R-124 vacuum evaporator, with a water bath setting at 25° C. to prevent possible freeze inside the flask. The stained microspheres are spun for 30 minutes in a centrifuge. The supernatant suspension is then passed through a funnel with a plug of glass wool, into storage bottle.

The excitation and emission spectra are measured and the percentage of solid beads determined. The beads are optionally coated with a 10% solution of Pluronic F-127 (Invitrogen Corp., P6866) and autoclave with deionized water to make the final microsphere suspension at 1% of solids in 2% of Pluronic F-127. The fluorescent Pluronic F-127 coated microspheres are stored at 4° C.

### Example 2

### Preparation of 2 μm Microspheres with NIR Emission (715 ex/755 em)

These fluorescent microspheres are prepared essentially as in Example 1, except that 1.1 mL of dye stock is added to the 10 ml tube and the microsphere stock is 2.0 μm sulfate polystyrene microspheres (8.1% solids, with surface charge content with surface charge content of 6 μEq/g (measured from conductometric titration)).

### Methods:

Studies of blood flow regulation have focused on the accurate description of the velocity field because other flow property calculations follow directly from these measurements. To obtain a detailed description of the velocity field in the microcirculation, conventional velocimetry techniques consider the field to be composed of a very large number of particles (Raffel et al., 2000; Westerweel, 1997). The spatial displacement of these particles in two separate images provides a measurement of the instantaneous in-plane velocity vector field (Adrian, 1984; Adrian, 1991). These instantaneous measurements provide a “snapshot” description of the velocity field. Commonly referred to as eulerian methods, these descriptions provide a highly resolved and computationally manageable velocity profile at a given point in space and moment in time.

In contrast to eulerian methods, lagrangian methods track the movement of individual particles. Particle tracking as a function of time provides a limited description of the velocity field at a particular point in space, but more information regarding the fate of individual particles. Thus, lagrangian descriptions may be especially useful in studies of leukocyte behavior in the microcirculation (Secomb et al., 2003; Su et al., 2003; West et al., 2001). Leukocyte trajectories that involve margination, mural interactions and prolonged residence times are best characterized using the computation of lagrangian coordinates.

The use of submicron fluorescent spheres, here termed nanoparticles, was investigated for microcirculatory particle tracking. The motion of the nanoparticles was investigated both in vitro and in vivo. Our findings suggest that intravital videomicroscopy and nanoparticles can provide accurate lagrangian flow description of microcirculation observed in vivo. D. J. Ravnic, et al. 4

Particles and red blood cells. The particles were manufactured with superior fluorescent characteristics and smaller size as compared with compositions reported previously (Bernard et al., 1996). The particles had low surface charge content: 6.2 uEq/gm (500 nm), 2.5 mEq/gm (2 um), and 1 uEq/gm (4 um). Although many different fluorescent colors were developed, the green (ex 488; em 510) and orange (ex 545 nm; em 570 nm) fluorescent nanoparticles were used for most experiments. Sheep red blood cells were obtained in a heparinized syringe and separated from white cells by density gradient centrifugation. The red cells were fluorescently labeled using the procedure included in the commercially available PKH26 Red Fluorescent Cell Linker kit (ex 551 nm; em 567 nm) (Sigma, St. Louis, Mo.). Based on empirical preliminary findings, the number of injected nanoparticles was normalized to the volume of red blood in 1 ml; namely, 452 um3. The number of injected nanoparticles was based on an equivalent total spherical volume: 6.912×109 500 nm particles, 1.08×108 2 um particles, and 1.3×107 4 um particles.

Electronic particle and cell volume. Electronic particle volume analysis was performed using a Coulter Z2 Particle Analyzer (Beckman Coulter, Miami, Fla.). The Coulter Z2, based on the Coulter principle (Ben-Sasson et al., 1974; Brecher et al., 1956), measured changes in electrical resistance produced by the nonconductive particles and cells suspended in a standard electrolyte solution (Isoton II; Beckman Coulter). A 50 um aperture was used with constant voltage settings. Particle minimum and maximum diameter settings were modified for the analysis. The particle size and number distributions were recorded over 256 channels and exported to Microsoft Excel (Redmond, Wash.) for statistical analysis. Flow cytometry. The nanoparticles and PKH26 labeled red blood cells were analyzed on an Epics XL (Beckman Coulter, Miami, Fla.) using gain settings calibrated to 4 peak Rainbow calibration particles (Spherotech, Libertyville, Ill.). The data was processed using WinList 5.0 (Verity, Topsham, Me.).

In vitro flow chamber. The design of the flow chamber has been previously described (Li et al., 1996; Li et al., 2001). Briefly, the chamber was machined from high-grade acrylic to minimize optical aberration and facilitate microscopy. Design features included 0.5 mm holes in both inlet and outlet manifold to rapidly stabilize laminar flow and permit the use of standard microscope slides. At each end of the flow deck, rounded fluid capacitors dampened eddy currents at the higher flows. The flow chamber was perfused with a NE-1000 withdrawal syringe pump (New Era, Farmingdale, N.Y.). In most experiments, the perfusate was normal saline containing either particles or red blood cells. D. J. Ravnic, et al.

Optical system. The optical systems were Nikon Eclipse TE2000 inverted epifluorescence microscopes using Nikon CFI Plan Fluor ELWD 10×, 20×, and 40× objectives. The intravital microscopy system used a Nikon Fluor WD 20× objective. An X-Cite (Exfo, Vanier, Canada) 120 watt metal halide light source and a liquid light guide was used to illuminate the tissue samples.

Excitation and emission filters (Chroma, Rockingham, Vt.) in separate LEP motorized filter wheels were controlled by a MAC5000 controller (Ludl, Hawthorne, N.Y.) and MetaMorph software 6.26 (Molecular Devices, Brandywine, Pa.). Electron multiplier CCD (EMCCD) camera. The flow chamber and intravital videomicroscopy 14-bit fluorescent images were digitally recorded with a EMCCD camera (C9100-02, Hamamatsu, Japan). The C9100-02 has a hermetic vacuum-sealed air-cooled head and on-chip electron gain multiplication (2000×). Images with 1000×1000 pixel resolution were routinely obtained at 30 fps; frame rates exceeding 60 fps were routinely obtained with 2×2 binning or subarray acquisition. The images were recorded in image stacks comprising 30 second to 10 minute video sequences.

Calculation of diffraction-limited resolution. Using matching apertures of the objective and condenser, the radius of the first order Airy diffraction ring was calculated using the formula 1.22 r=λ/2 NA, where λ is the wavelength and NA is the numerical aperture of the objective. The minimum resolved distance between Airy patterns (Rayleigh criterion) was calculated where r=0.61λ/NA and used to determine maximum concentration of the particles in flow chamber experiments. The concentration of red cells and particles was, at maximum, 10-fold less than the concentration defined by the Rayleigh criterion. Quantitative morphometry. Out-of-focus blurring and camera-dependent streaking was assessed using quantitative image analysis (MetaMorph; Molecular Devices, Downingtown, Pa.).

The out-of-focus effects were assessed by optical dispersion. The optical dispersion was a measure of the total fluorescence intensity around the centroid of the particle. The camera-dependent streaking was assessed by the elliptical form factor. The elliptical form factor was calculated as the ratio of a particle's breadth to its length. Mice. Male Balb/c mice (Jackson Laboratory, Bar Harbor, Me.), 25-33 g, were used in all experiments.

Multi-frame particle tracking. Particle tracking was performed on digitally recorded and distance calibrated multi-image “stacks.” The image stacks produced a sequential time history of velocity and direction as the acquired images were time stamped based on the 100 mhz system bus clock of the Xeon processor (Intel, Santa Clara, Calif.). The movement of individual particles D. J. Ravnic, et al. was tracked using the MetaMorph (Molecular Devices) object tracking applications. The intensity centroids of the particles were identified and their displacements tracked through planes in the source image stack. For displacement reference, the algorithm used the location of the particle at its first position in the track. Each particle was imaged as a high contrast fluorescent disk and its position was determined with sub pixel accuracy. The image of the particle was tracked using a cross correlation centroid-finding algorithm to determine the best match of the cell position in successive images. The resulting measures included the X and Y coordinates, velocity, mean displacement, mean vector length, mean angle (the angle of the mean vector of the object), and the angular deviation.

Ear microscopy. The ear intravital microscopy was performed by using a custom machined titanium stage (Miniature Tool and Die, Charlton, Mass.). The tissue contact area consisted of a 2-mm vacuum gallery that provided tissue apposition to the stage surface without compression of the tissue and with minimal circulatory disturbances.

Organ distribution. The relative organ distribution of the fluorescent particles was determined using modified procedures developed previously (Luchtel et al., 1998; Powers et al., 1999; Van Oosterhout et al., 1995). Briefly, tissue samples were harvested from the animals and digested in 8 mL of 4N KOH for 96 hrs. The fluorescence was separated by individually filtrating the digested samples through a 0,4-μm-pore polycarbonate filter (Whatman Inc. Florham Park, N.J.) with negative pressure. The filters containing the fluorescence were rinsed with potassium phosphate buffer (pH 7.0) and followed with deionized water. Each filter was air-dried and then soaked in 4 ml of 2-ethoxyethyl acetate (Cellosolve® acetate, Aldrich Chemical, Milwaukee, Wis.) for 4 days. 2 mL of supernatant was then transferred into a cuvette and read in a fluorescence spectrophotometer (Hitachi F-4500) at the dye-specific excitation and emission wavelengths. D. J. Ravnic, et al. 7

### Results

Particle fluorescence intensity. The particles used in this study were hydrophobic microspheres of 3 diameters: 500 nm, 2 um and 4 um. The volumes of the 4 um diameter particles were comparable to the volume of sheep red blood cells when assessed by electrical impedance. To compare the fluorescence intensity of the particles to a standard blood tracer, particles of 0.5 um, 2 um and 4 um were compared to PKH26 labeled red blood cells (RBC) by flow cytometry. The particles demonstrated a 500-fold to 10.000-fold increase in fluorescence intensity over the red blood cells labeled with the lipophilic dye PKH26. When normalized for volume, the particles demonstrated a 20,0000 to 28,000-fold greater fluorescence intensity per unit volume when compared to the fluorescently labeled red cells.

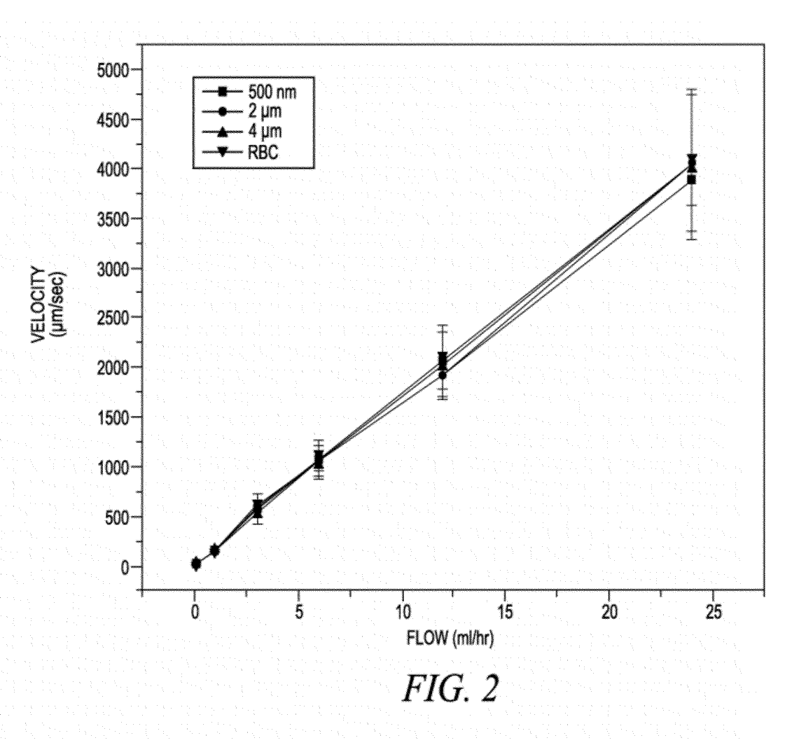

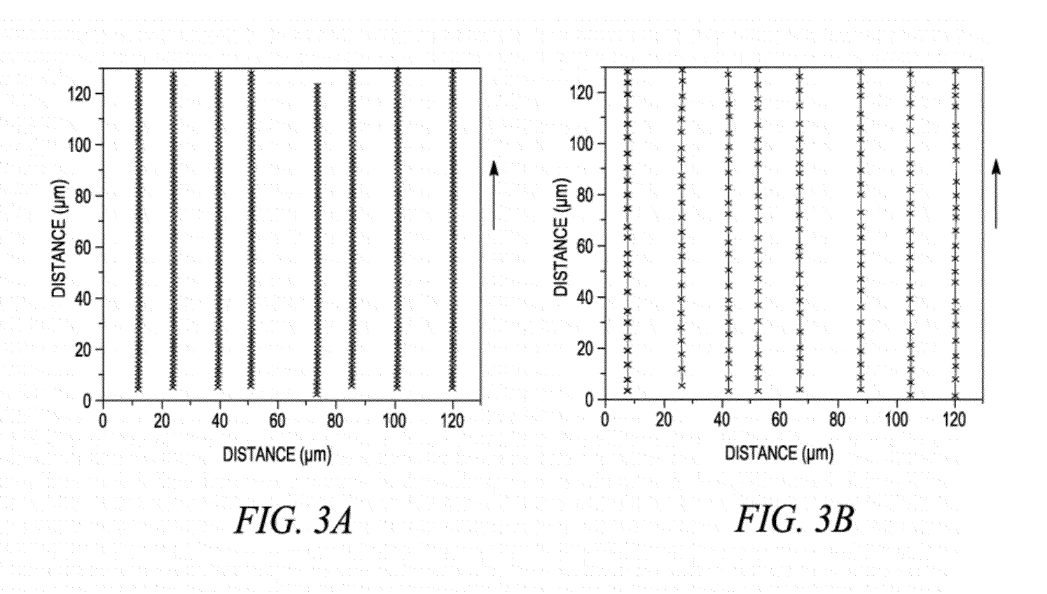

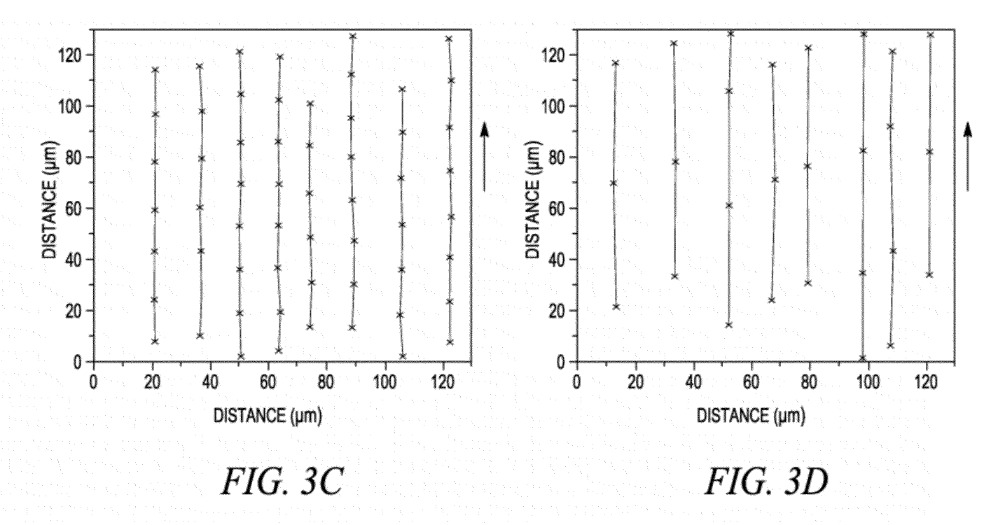

Tracking particles in vitro. To assess the resolution of the tracer particles at velocities comparable to those observed in vivo, (Johnson and Wayland, 1967) we tracked the 500 nm, 2.0 um and 4.0 um particles as well as PKH26 labeled RBCs in a flow chamber at velocities from 125 um/sec to 4000 um/sec. As expected, the measured particle velocities demonstrated increasing variance at increased flow velocities (FIG. 2). The information content of particle tracking was dependent on flow velocity, the size of the optical field, and the acquisition rate of the CCD camera (FIG. 3).

Morphometry of particle images. In addition to the diffraction limitations of the optical system (Olsen and Adrian, 2000), the sampling error at higher velocities reflects out-of-focus effects and CCD camera-dependent streaking artifact. To assess out-of-focus effects, we measured the optical dispersion of the particles at different velocities. These morphometric features demonstrated over sampling of the particle image only at velocities of 4000 um/sec (p<0.05).

To assess the characteristic fluorescence streaking on EMCCD acquired images, we assessed the elliptical form factor of the particle images. Elliptical form factor, a ratio of particle breadth to length, demonstrated increased streaking at 4000 um/sec, (p<0.05), but no significant difference at lower velocities.

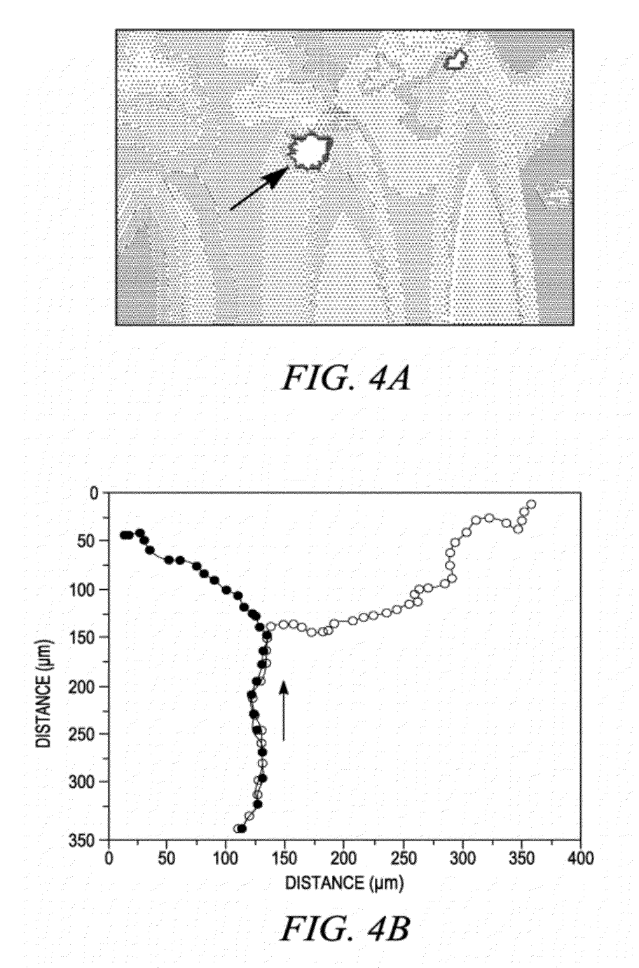

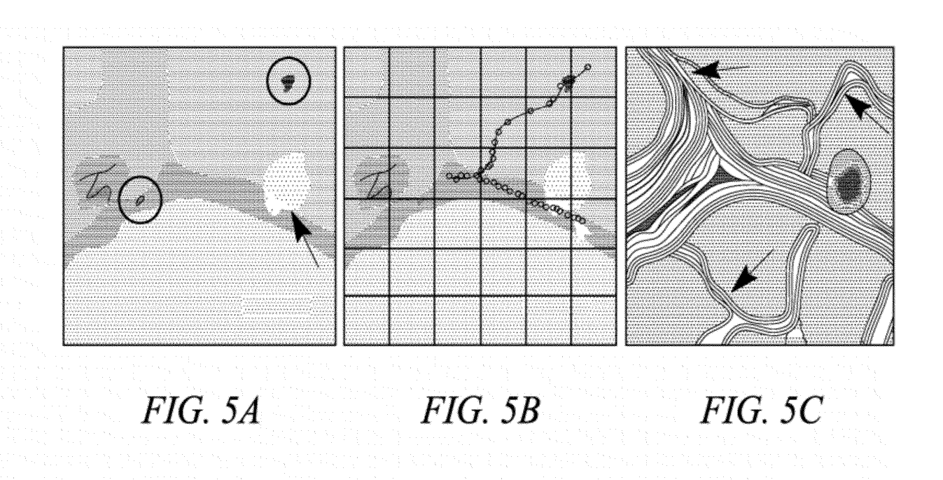

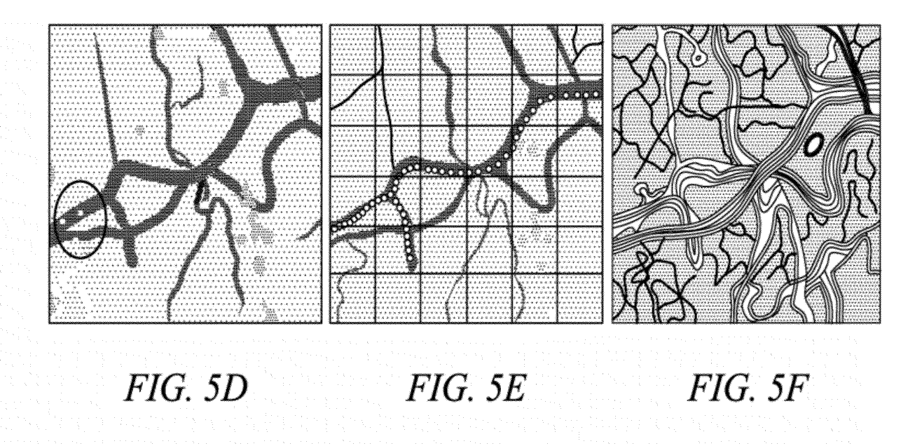

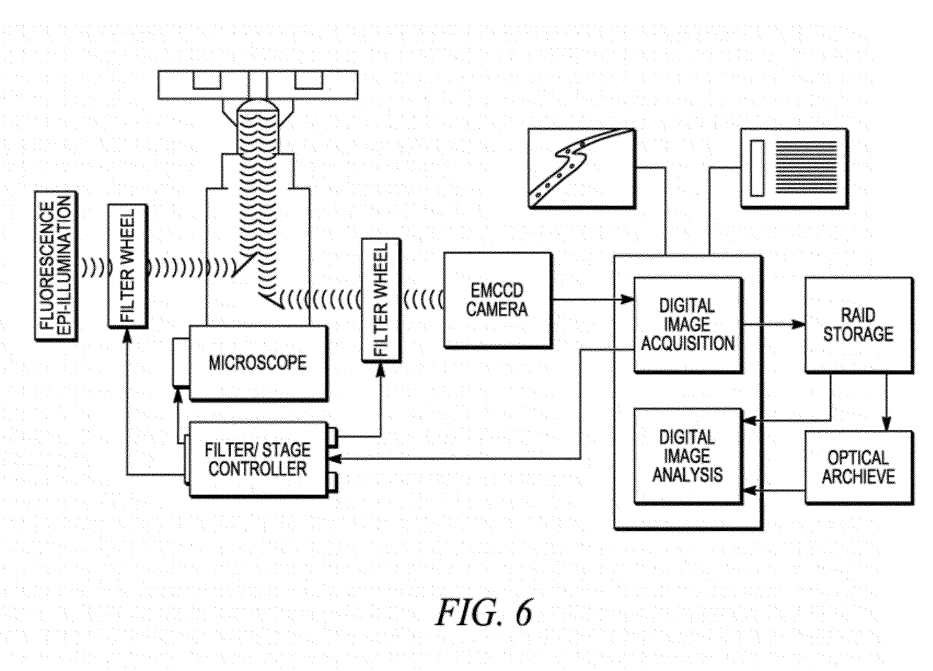

Particle detection in vivo. To assess sampling of particle velocity within the microcirculation, empirically defined concentrations of particles were injected into the mouse tail vein and the ear microcirculation was evaluated by intravital microscopy. The particles were readily tracked in the mouse ear microcirculation (FIG. 4). Continuous monitoring of the ear microcirculation demonstrated that the circulation half-life (t_{1/2}) was inversely related to particle size. The videomicroscopy demonstrated a progressive increase in the number of static particles within the microscopy window at all three particle sizes; however, the prevalence of static particles was greatest with the 4 um particles. Organ harvest at the conclusion of the experiment demonstrated that greater than 50% of the particles were retained in the liver irrespective of particle size.

While particular embodiments of the invention have been illustrated and described, it will be appreciated that various changes can be made therein without departing from the spirit and scope of the invention.

1. A method for measuring blood flow in a subject wherein the method comprises:
administering to the subject a plurality of microspheres, wherein the microspheres are impregnated with a dye having an excitation and emission spectrum compatible with in vivo or intravital imaging and further, wherein the microspheres have a hydrophobic outer surface;
illuminating microspheres within the subject with an appropriate wavelength to form illuminated microspheres; and
observing the illuminated microspheres;
wherein, the blood flow is measured by detecting the movement of the microspheres in the subject.

2. The method of claim 1, wherein velocity of the blood flow is determined.

3. The method of claim 1, wherein the plurality of microspheres are comprised of a first population having a substantially uniform first diameter.

4. The method of claim 3, wherein the plurality of microspheres are comprised of a first population having a substantially uniform first diameter and a second population having a substantially uniform second diameter.

5. The method of claim 4, wherein the first population is impregnated with a different dye than the second population.

6. The method of claim 4, wherein the first diameter is different than the second diameter.

7. The method of claim 6, wherein the first diameter is 0.1 μm to 0.75 μm and the second diameter is 0.76 μm to 2 μm.

8. The method of claim 1, wherein said microspheres have a diameter of about 0.1 μm to 4 μm.

9. The method of claim 1, wherein said microspheres have a diameter of about 0.2 μm to 2 μm.

10. The method of claim 1, wherein said microspheres have a surface charge of less than 50 μEq/gram.

11. The method of claim 1, wherein said microspheres have a surface charge of less than 10 μEq/gram.

12. The method of claim 3, wherein said first population has a diameter variation of 5% or less.

13. The method of claim 3, wherein said first population has a diameter variation of 2% or less.

14. The method of claim 1, wherein said hydrophobic outer surface is comprised of an organic polymer.

15. The method of claim 14, wherein said hydrophobic outer surface is comprised of polymerized styrene moieties.

16. The method of claim 14, wherein the organic polymer is substituted with sulfate groups.

17. The method of claim 1, wherein said hydrophobic outer surface is a block co-polymer.

18. The method of claim 17, wherein said block co-polymer is comprised of polyoxyethylene (PEO) and polyoxypropylene (PPO) moieties.

19. The method of claim 1, wherein said microspheres are substantially free of aggregates.

20. The method of claim 1, wherein said microspheres are substantially free of aggregation with at least one of the following: leukocytes, erythrocytes, thrombocytes, serum proteins, electrolytes, carbohydrates, fats, or minerals.

21. The method of claim 1, wherein said microspheres are administered to the subject parenterally.

22. The method of claim 1, wherein the dye is selected from the group consisting of a pyrene, an anthracene, a naphthalene, an acridine, a stilbene, an indole, an oxazole, benzoxazole, a thiazole, a benzothiazole, a 4-amino-7-nitrobenz-2-oxa-1,3-diazole (NBD), a carbocyanine, a carbostyryl, a porphyrin, a salicylate, an anthranilate, an azulene, a perylene, a pyridine, a quinoline, a xanthene, a borapolyazaindacine, an oxazine, a benzoxazine, a carbazine, a phenalenone, a coumarin, a benzofuran, a benzphenalenone, a semiconductor nanocrystal, and a fluorescent protein.

23. The method of claim 1, further comprising the step of incubating said subject for a sufficient amount of time for the microspheres to circulate prior to illuminating the microspheres.

24. The method of claim 1, wherein the microspheres are used to monitor the pattern or turbulence of blood flow in the subject.

25. The method of claim 1, wherein the subject is suffering from a disease associated with obstructed or abnormal blood flow.

26. The method of claim 25, wherein the disease is selected from a viral infection, a bacterial infection, heart disease, cancer, ischemia, autoimmune disease, a CNS disorder, a metabolic disease, or a respiratory disease.

28. (canceled)
